SEQUENCE LISTING (2 sequences)

SEQ ID NO: 1 - protein, 7 aa, Artificial Sequence
  synthetic peptide
  MISC_FEATURE (1)..(1): XAA = the absence of an amino acid or L-Tyr
  MISC_FEATURE (2)..(2): XAA = one of the amino acids L-Leu, L-Ala or D-Ala
  MISC_FEATURE (2)..(2): XAA = one of the amino acids L-Leu, L-Ala or D-Ala
  MISC_FEATURE (3)..(3): XAA = one of the amino acids L-His, D-His, L-Ala or D-Ala
  MISC_FEATURE (6)..(6): XAA = one of the amino acids L-Gln, L-Ala or D-Ala
  MISC_FEATURE (7)..(7): C-terminal is OMe or NH2
XXXKLXT

SEQ ID NO: 2 - protein, 7 aa, Artificial Sequence
  syntetic peptide
  MISC_FEATURE (1)..(1): D-Thr
  MISC_FEATURE (2)..(2): XAA = one of the amino acids D-Gln, D-Ala or L-Ala
  MISC_FEATURE (3)..(3): D-Leu
  MISC_FEATURE (4)..(4): D-Lys
  MISC_FEATURE (5)..(5): XAA = one of the amino acids D-His, L-His, D-Ala or L-Ala
  MISC_FEATURE (6)..(6): XAA = one of the amino acids D-Leu, D-Ala or L-Ala
  MISC_FEATURE (7)..(7): XAA = the absence of an amino acid or D-Tyr
  MISC_FEATURE (7)..(7): C-terminal is OMe or NH2
TXLKXXX

(19) United States
(12) Patent Application Publication
(10) Pub. No.: US 2021/0228677 A1
(43) Pub. Date: Jul. 29, 2021

(54) PHARMACEUTICAL COMPOSITION AND A METHOD FOR ITS MANUFACTURE

(71) Applicants: Oleg Arkadyevich KOTIN, Gatchina (RU); Pharmbiotech OY, Kotka (FI)

(72) Inventors: Oleg Arkadyevich KOTIN, Gatchina (RU); Arkadiy Mihaylovich KOTIN, St. Petersburg (RU); Maksim Olegovich EMELYANOV, St. Petersburg (RU)

(21) Appl. No.: 17/157,592

(22) Filed: Jan. 25, 2021

Related U.S. Application Data
(60) Provisional application No. 62/966,715, filed on Jan. 28, 2020.

(51) Int. Cl. A61K 38/08; A61K 47/10; A61P 29/02

(52) U.S. Cl. CPC A61K 38/08; A61P 29/02; A61K 47/10

(57) ABSTRACT

Disclosed is a storage stable pharmaceutical composition containing at least one active pharmaceutical ingredient selected from peptides of the general formula 1 [SEQ ID NO: 1-144] or peptides of the general formula 2 [SEQ ID NO: 145-288], or, their derivatives, analogs or pharmaceutically acceptable salts, or co-crystals of these peptides, their derivatives or analogues; and a pharmaceutically acceptable carrier, wherein the composition retains at least 80% of the potency of active pharmaceutical ingredient in the pharmaceutical composition after storage for at least four months at room temperature. Also, disclosed are process for their preparation and methods of use.

### CROSS-REFERENCE TO RELATED APPLICATIONS

This application claims priority to and the benefit of U.S. Provisional Patent Application Ser. No. 62/966,715, filed Jan. 28, 2020, the subject matter of which is incorporated herein by reference.

### INCORPORATION BY REFERENCE OF SEQUENCE LISTING

The Sequence Listing in an ASCII text file, named REVISED SEQUENCE LISTING.txt of 2 KB, created on Mar. 12, 2021, and submitted to the United States Patent and Trademark Office via EFS-Web, is incorporated herein by reference.

### FIELD

The present specification relates to stable pharmaceutical compositions that contain at least one peptide, its derivatives, analogue, or pharmaceutically acceptable salt, and co-crystals of these peptides, their derivatives or analogues. In particular, the present specification relates to storage-stable pharmaceutical compositions and methods for preparing such compositions. The compositions are particularly suitable for parenteral administration and for the treatment of pain.

### BACKGROUND

The group of synthetic low-molecular peptides with non-narcotic type of analgesia of the general formula 1 [SEQ ID NO: 1-144] and 2 [SEQ ID NO: 145-288], hereinafter referred to as “developed synthetic peptides”, was developed by modifying the minimum salmon calcitonin molecule fragment sCT₁₆₋₂₁, which retains the previously reported analgesic activity of the full-sized molecule [1].

It was found that the developed synthetic peptides with a non-narcotic type of analgesic effect, effective for various methods of parenteral administration (suboccipital, intramuscular, intranasal and intravenous) on various models of tonic and acute pain, are specific clathrin inhibitors [2].

Inhibition of clathrin function has a key role in the prevention or treatment of a number of diseases and conditions, the list of which includes, but is not limited to, cell proliferative diseases and conditions, multifocal leukoencephalopathy, polycystic kidney disease, diseases associated with R-amyloid (such as Alzheimer's disease), neurodegenerative disorders, neuropsychiatric disorders, psychotic disorders, psychoses, bipolar disorders, schizophrenia, aberrant unregulated excited neurons, convulsions, neuropathic pain, migraines, as well as all diseases and conditions mediated or otherwise associated with the transmission of the synaptic signal involving the processes of clathrin-mediated endocytosis (CME) (for example, such as epilepsy) or with impaired recirculation of cell vesicles. The list of diseases and conditions in which inhibition of clathrin function has a key role is indicated earlier in the patent [2], the content of Table 1 of which is incorporated herein by reference in their entirety.

It was also shown that the developed synthetic peptides more effectively reduce the manifestation of signs of pain than ketorolac tromethamine, which is comparable with morphine in analgesic activity and significantly superior to other NSAIDs [3].

However, usually peptide-based pharmaceutical compositions are inherently unstable due to their susceptibility to chemical (e.g., oxidation or hydrolysis) and physical (e.g., aggregation, deposition or adsorption on the surface) degradation [4].

Such susceptibility to degradation during prolonged storage at room temperature leads to the fact that the recommended storage conditions for a commercially available product require storage in a cold chain with a temperature range of 2° C. to 8° C. These special storage requirements, therefore, lead to an increase in the cost of the final product when it reaches the final consumer.

In particular, FIG. 1 shows that without special measures by doing stability, a pharmaceutical composition that contains one of the above-mentioned “developed synthetic peptides” degrades in four months of storage at a temperature of 25° C.

There is a need to develop stable and storage stable pharmaceutical compositions suitable primarily for parenteral administration that contain developed synthetic peptides or their derivatives, or their analogs, or pharmaceutically acceptable salts and co-crystals of these peptides, their derivatives and analogues, as well as a method for producing such compositions.

Since the properties of peptide molecules strongly depend on their structure, and the family of “developed synthetic peptides” was obtained by the authors of the present invention for the first time, information on the stability of pharmaceutical compositions with just such peptides during storage at room temperature was unknown.

Patent publication [5] proposes a liquid pharmaceutical composition comprising a calcitonin peptide as an active ingredient and specific concentrations of citric acid or its salt as a stabilizer and absorption enhancer. However, the “developed synthetic peptides” differ from the peptides indicated in the mentioned patent publication. In addition, the stability conditions of the pharmaceutical composition in the publication [5] do not provide for storage for at least four months at room temperature.

Patent publication [6] proposes a stable, non-aqueous and ready-to-use injection composition of a peptide drug or its pharmaceutically acceptable salt or co-crystal, containing the peptide drug or its pharmaceutically acceptable salt or co-crystal and non-aqueous solvent system. However, the physiological acceptability of non-aqueous solvents is usually less than that of aqueous, and therefore, the preferred solvent is water with or without the addition of organic solvents [7]. In addition, the stability conditions of the pharmaceutical composition in the publication [6] do not provide for storage for at least four months at room temperature.

Patent publication [8] proposes an aqueous parenteral pharmaceutical composition for improving the stability of a polypeptide comprising a polypeptide and glycerin. However, the stability conditions of the pharmaceutical composition in the publication [8] do not provide for storage for at least four months at room temperature.

There are numerous examples of other methods that are used to increase stability and improve storage conditions of peptide pharmaceutical compositions. All of them have certain disadvantages that narrow the possibilities of their use in medicine.

There is a need in the art for storage stable pharmaceutical compositions, mainly intended for parenteral administration, which contain developed synthetic peptides or their derivatives or their analogues or pharmaceutically acceptable salts and co-crystals of these peptides, their derivatives and analogues. In addition, there is a need in the art for a process for preparing such storage stable pharmaceutical compositions.

### SUMMARY OF INVENTION

The inventors of the present invention have surprisingly found that it is possible to develop storage stable, for at least four months at room temperature, pharmaceutical composition comprising at least one active pharmaceutical ingredient, selected from developed synthetic peptides or their derivatives or their analogues or pharmaceutically acceptable salts and co-crystals of these peptides, their derivatives and analogues and a pharmaceutically acceptable carrier, wherein the composition may not include special stabilizing agents.

In particular, the present inventors have found that such stable pharmaceutical composition, containing the peptides disclosed herein, may be obtained by

a) using pharmaceutically acceptable carrier, comprising entirely of a solvent, wherein the composition retains at least 80% of the potency of active pharmaceutical ingredient in the pharmaceutical composition after steam sterilization;

b) using pharmaceutically acceptable carrier, containing a specific solvent and, in addition to the solvent at least one more ingredient, wherein the at least one or more ingredient is a pH adjusting agent; isotonicity agent; preservative; chelating agent; antioxidant; surfactant; viscosifier or absorption promoter;

c) using a combination of methods a) and b), depending on the structure of the active ingredient peptide.

Further, the inventors of the present invention have found that that the resulting pharmaceutical composition may remain stable when subjected to the stability condition and can be stored at room temperature over the storage period for at least four months and can avoid requiring cold chain storage.

The inventors have also proposed a simple and cost-effective way to obtain storage stable pharmaceutical compositions.

In one aspect, the specification relates to a storage stable pharmaceutical composition comprising at least one active pharmaceutical ingredient selected from peptides of the general formula 1 [SEQ ID N: 1-144] or peptides of the general formula 2 [SEQ ID NO: 145-288] or their derivatives or their analogues or pharmaceutically acceptable salts and co-crystals of the above peptides, their derivatives and analogues, and a pharmaceutically acceptable carrier, wherein the composition retains at least 80% of the potency of active pharmaceutical ingredient in the pharmaceutical composition after storage for at least four months at room temperature.

In another general aspect, there is provided storage stable pharmaceutical composition comprising developed synthetic peptides or their derivatives or their analogues or pharmaceutically acceptable salts and co-crystals of the above peptides, their derivatives and analogues, and a pharmaceutically acceptable carrier, wherein the composition when stored at 25° C. in closed vials for four months, the total amount of related substances does not increase more than 15% by weight to the active pharmaceutical ingredient.

In one embodiment, for example and without limitation, the pharmaceutically acceptable carrier contains a solvent and pH adjusting agent, and has a pH of 5.0±0.1. In another embodiment, for example and without limitation, pharmaceutically acceptable carrier contains a solvent, pH adjusting agent, isotonicity agent, preservative, chelating agent, antioxidant, surfactant, viscosifier, absorption promoter and has pH 3.7±0.1. In another embodiment, for example and without limitation, the steam sterilized pharmaceutically acceptable carrier contains only a solvent and has pH 7.0±0.1. In another embodiment, for example and without limitation, pharmaceutically acceptable carrier contains a solvent, pH adjusting agent, isotonicity agent and has pH 4.0±0.1.

In a further aspect, there is provided storage stable pharmaceutical composition comprising developed synthetic peptides or their derivatives or their analogues or pharmaceutically acceptable salts and co-crystals of the above peptides, their derivatives and analogues, and a pharmaceutically acceptable carrier. In one embodiment, for example and without limitation, the composition retains at least 80% of the potency of active pharmaceutical ingredient in the pharmaceutical composition after steam sterilization. In another embodiment, for example and without limitation, the total amount of related substances in the composition does not increase more than 15% by weight to the active pharmaceutical ingredient after steam sterilization.

In still another aspect, there is provided storage stable pharmaceutical composition comprising developed synthetic peptides or their derivatives or their analogues or pharmaceutically acceptable salts and co-crystals of the above peptides, their derivatives and analogues, and a pharmaceutically acceptable carrier, wherein the derivative is a peptide of five to fourteen amino acid residues containing an amino acid sequence that has at least 50% amino acid sequence identity in length relative to the amino acid sequence of peptides of the general formula 1 [SEQ ID NO: 1-144] or 2 [SEQ ID NO: 145-288], which retains peptide activity and/or interacting with the same specific receptor, and may differ from these sequences by truncation, deletion, substitution, addition or modification of one or more amino acids selected from a naturally occurring amino acid, a natural non-protein amino acid, a non-natural amino acid, or a modified or unusual amino acid residue.

In another aspect, there is provided storage stable pharmaceutical composition comprising developed synthetic peptides or their derivatives or their analogues or pharmaceutically acceptable salts and co-crystals of the above peptides, their derivatives and analogues, and a pharmaceutically acceptable carrier, wherein the pharmaceutically acceptable carrier comprises a solvent, wherein the solvent is water, ethylene glycol, diethylene glycol, triethylene glycol, tetraethylene glycol, polyethylene glycol, propylene glycol, dipropylene glycol, tripropylene glycol, methoxypropylene glycol, polyethylene oxide, glycerin, ethanol, propyl alcohol, isopropyl alcohol, benzyl alcohol, benzyl benzoate, glycofurol, or combinations thereof.

In some embodiments, for example and without limitation, the pharmaceutically acceptable carrier consists entirely of a solvent, wherein the solvent is water, ethylene glycol, diethylene glycol, triethylene glycol, tetraethylene glycol, polyethylene glycol, propylene glycol, dipropylene glycol, tripropylene glycol, methoxypropylene glycol, polyethylene oxide, glycerin, ethanol, propyl alcohol, isopropyl alcohol, benzyl alcohol, benzyl benzoate, glycofurol, or combinations thereof. In particular embodiments, where pharmaceutically acceptable carrier consists entirely of a solvent, storage stability, as disclosed herein, is obtained by steam sterilization.

In some embodiments, for example and without limitation, the pharmaceutically acceptable carrier comprises at least one more ingredient, wherein the one or more ingredient is a pH adjusting agent; isotonicity agent; preservative; chelating agent; antioxidant; surfactant; viscosifiers or absorption enhancer.

In another aspect, there is provided storage stable pharmaceutical composition wherein the pH is from about 2.0 to about 11.0. In one embodiment, for example and without limitation, the storage stable pharmaceutical composition has a pH from about 2.0 to about 9.0. In another embodiment, for example and without limitation, the storage stable pharmaceutical composition has a pH from about 2.5 to about 7.0. In another embodiment, for example and without limitation, the storage stable pharmaceutical composition has a pH from about 3.0 to about 7.0. In another embodiment, for example and without limitation, the storage stable pharmaceutical composition has a pH from about 3.0 to about 5.0. In another embodiment, for example and without limitation, the storage stable pharmaceutical composition has a pH about 4.0.

In another aspect, there is provided storage stable pharmaceutical composition wherein the concentration of active pharmaceutical ingredient is in the range of 1 μg/ml to 500 mg/ml.

In another aspect, there is provided storage stable pharmaceutical composition wherein the concentration of active pharmaceutical ingredient is in an amount sufficient to reduce the number of manifestations of pain symptoms when the pharmaceutical composition is administered to a subject in need thereof.

In another general aspect, there is provided a method of treating moderate to moderately severe pain that requires analgesia at the opioid level in a subject in need thereof, comprising administering to the subject a storage stable pharmaceutical composition comprising at least one active pharmaceutical ingredient selected from peptides of the general formula 1 [SEQ ID NO: 1-144] or 2 [SEQ ID NO: 145-288], or their derivatives, or their analogues, or pharmaceutically acceptable salts and co-crystals of these peptides, their derivatives and analogues; and a pharmaceutically acceptable carrier, wherein the composition retains at least 80% of the potency of active pharmaceutical ingredient in the pharmaceutical composition after storage for at least four months at room temperature.

Also, contemplated are uses of the active pharmaceutical ingredients disclosed herein.

In some embodiments there is provided a process of preparing a storage stable pharmaceutical composition, wherein the process comprises, but not limited to, a steam sterilization stage that provides improved stability.

In some embodiments there is provided a process of preparing a storage stable pharmaceutical composition, wherein the process comprising, but not limited to, the following stages:

(a) the preparation of a pharmaceutically acceptable carrier,

(b) adding to the carrier at least one active pharmaceutical ingredient;

(c) optionally adding at least one more ingredient selected from the group consisting of a pH adjusting agent; isotonicity agent; preservative; chelating agent; antioxidant; surfactant; viscosifier or absorption promoter;

(d) filling the pharmaceutical composition in a suitable container;

(e) steam sterilization.

The details of one or more embodiments of the invention are set forth in the description below. Other features, objects, and advantages of the invention will be apparent from the description.

### BRIEF DESCRIPTION OF THE DRAWINGS

Reference will now be made, by way of example, to the accompanying drawings which show example embodiments of the present application, and in which:

FIG. 1 is a chromatogram of a composition, similar to pharmaceutical composition No. 1, but without special measures for stability, after preparation (A) and after four months of storage at room temperature 25° C. (B). The amount of active pharmaceutical ingredient after four months of storage was 50.1% of the initial value, the amount of related impurities exceed 49%;

FIG. 2 is a chromatogram of steam sterilized pharmaceutical composition No. 1 after preparation (A) and after four months of storage at room temperature 25° C. (B). The amount of active pharmaceutical ingredient after four months of storage was 98.3% of the initial value, the amount of related impurities did not exceed 1.5%;

FIG. 3 is a chromatogram of pharmaceutical composition No. 2 after preparation (A) and after four months of storage at room temperature 25° C. (B). The amount of active pharmaceutical ingredient after four months of storage was 97.7% of the initial value, the amount of related impurities did not exceed 2.0%;

FIG. 4 is a chromatogram of pharmaceutical composition No. 3 after preparing (A) and after steam sterilization (B). The amount of active pharmaceutical ingredient after steam sterilization was 97.1% of the initial value, the amount of related impurities did not exceed 2.7%.

FIG. 5 shows the values of the excitation threshold of painful receptors for tooth pulp in animals, leading to the corresponding level of behavioral reaction, 60 minutes after suboccipital administration of physiological saline (K), tramadol hydrochloride at a dose of 12 mg/kg (R), pharmaceutical composition 7 at a dose of 180 μg/kg (T1), pharmaceutical composition 8 at a dose of 180 μg/kg (T2), pharmaceutical composition 9 at a dose of 180 μg/kg (T3), for the canine (A) and molar (B), respectively (I; II; III—levels of manifestation of pain reaction).

Similar reference numerals may have been used in different figures to denote similar components.

### DESCRIPTION OF EXAMPLE EMBODIMENTS

It should be understood that the detailed description and specific examples, while indicating embodiments of the invention, are given by way of illustration only, since various changes and modifications within the spirit and scope of the invention will become apparent to those skilled in the art. One skilled in the art, based upon the definitions herein, may utilize the present invention to its fullest extent. The following specific embodiments are to be construed as merely illustrative, and not limitative of the remainder of the disclosure in any way whatsoever.

Unless otherwise defined, all the terms used herein, including the technical and scientific terms, have the meaning as that generally understood by one of ordinary skill in the art to which the present invention relates.

For the purpose of the disclosure, listed below are definitions of various terms used to describe the present invention. Unless otherwise indicated, these definitions apply to the terms as they are used throughout the specification and the appended claims, either individually or as part of a larger group. They should not be interpreted in the literal sense. They are not general definitions and are relevant only for this application.

It should be noted that, as used in this specification and the appended claims, the singular forms “a”, “an” and “the” include plural referents unless the content clearly dictates otherwise.

It should be noted that the term “or” is generally employed in its sense including “and/or” unless the content clearly dictates otherwise.

As used herein, the term “about” means approximately and in the context of numerical values the term “about” can be construed to estimate a value that is +10% of the value or range recited.

As used herein, the term “pharmaceutical composition” or “composition” means a single unit dose or multiple dose of an active pharmaceutical ingredient and a pharmaceutically acceptable carrier, which can be prepared by the methods described in one or more embodiments of the present invention.

For the purposes of the present invention, the pharmaceutical composition is preferably placed in a suitable container. This container may also be part of a device in which a pharmaceutical composition is prepared immediately prior to administration to a patient.

Various kinds of the pharmaceutical composition of the present invention can be selected from the group which includes, but is not limited to: solution, suspension, syrup, liquid, gel, hydrogel, emulsion, liposome, aerosol, fog, film, polymer, implant or sustained release composition.

The method of administering to a subject a pharmaceutical composition of the present invention may be selected from the group which includes, but is not limited to: transdermally, intravenously, dermally, subcutaneously, nasal, inhalation, intramuscularly, intraperitonealy, orally, intracranially or subocciptally.

In some embodiments, the composition is administered to a subject once a day, twice a day, three times a day, or more often. In other embodiments, the invention is administered every other day or less.

As used herein, the term “pharmaceutically acceptable” means suitable for normal pharmaceutical use, that is, without causing side effects in patients.

As used herein, the term “carrier” refers to a pharmaceutically acceptable substance that can be administered to a patient together with the active pharmaceutical ingredient of this invention and which does not decrease pharmacological activity of active pharmaceutical ingredient.

As used herein, the term “storage stable” or “stable” means that when the pharmaceutical composition is stored at room temperature (25° C.) in closed vials for an extended period of time, for example four months, there is no significant change in the dissolution profile and/or therapeutically effective amount and/or said composition has chromatographic purity when the identified impurities are within acceptable limits.

In particular, as is known to one skilled in the art, peptide compositions are considered stable when, after four months of storage at 25° C. or, equivalently, after two months of storage at 37° C., at least about 65% of the active pharmaceutical ingredient remains chemically and physically stable [9, 10].

The term “chemical stability” means that the percentage of decomposition products formed by chemical pathways, such as oxidation or hydrolysis, is acceptable. In particular, the drug is considered chemically stable if after four months of storage at a temperature of 25° C. or, equivalently, after two months of storage at a temperature of 37° C., no more than approximately 20% (by weight to the active pharmaceutical ingredient) of decay products are formed.

The term “physical stability” means that the percentage of fibrils and aggregates is acceptable. In particular, the drug is considered physically stable if, after four months of storage at a temperature of 25° C. or, equivalently, after two months of storage at a temperature of 37° C., no more than approximately 15% (by weight to the active pharmaceutical ingredient) of fibrils and aggregates are formed.

A change in the content of a chemically and physically stable active pharmaceutical ingredient in the pharmaceutical composition results in a corresponding change in the potency (specific activity) of this active pharmaceutical ingredient in the pharmaceutical composition and a corresponding change in the therapeutically effective amount of the pharmaceutical composition.

As used herein, the term “therapeutically effective amount” or “therapeutically effective dose” means the amount of the pharmaceutical composition that, when administered to a subject to treat a disease, disorder or other undesirable pathological condition, is sufficient to provide a beneficial effect on the disease, disorder or condition.

Particularly preferred are peptide compositions that retain at least about 80% of the potency of active pharmaceutical ingredient in the pharmaceutical composition after four months of storage at 25° C. Even more preferred are peptide compositions that retain at least about 80% of the potency of active pharmaceutical ingredient in the pharmaceutical composition after sterilization, or peptide compositions wherein the total amount of related substances in the composition does not increase more than 15% by weight to the active pharmaceutical ingredient after steam sterilization.

The specification relates to a storage stable pharmaceutical composition which comprises at least one active pharmaceutical ingredient as defined above and a pharmaceutically acceptable carrier, wherein the composition retains at least 80% of the potency of active pharmaceutical ingredient in the pharmaceutical composition after storage for at least four months at room temperature. In one embodiment, for example and without limitation, composition retains at least 85% of the potency of active pharmaceutical ingredient in the pharmaceutical composition after storage for at least four months at room temperature. In another embodiment, for example and without limitation, composition retains at least 90% of the potency of active pharmaceutical ingredient in the pharmaceutical composition after storage for at least four months at room temperature. In another embodiment, for example and without limitation, composition retains at least 95% of the potency of active pharmaceutical ingredient in the pharmaceutical composition after storage for at least four months at room temperature.

The specification further relates to a storage stable pharmaceutical composition which comprises at least one active pharmaceutical ingredient as defined above and a pharmaceutically acceptable carrier, wherein the composition when stored at 25° C. in closed vials for four months, the total amount of related substances does not increase more than 15% by weight to the active pharmaceutical ingredient. In one embodiment, for example and without limitation, the total amount of related substances does not increase more than 10% by weight to the active pharmaceutical ingredient. In another embodiment, for example and without limitation, the total amount of related substances does not increase more than 5% by weight to the active pharmaceutical ingredient.

The term “related substances” means substances that are structurally related to the active pharmaceutical ingredient. These substances may be decomposition products or impurities arising during the manufacturing process or during storage of the pharmaceutical composition.

In a specific embodiment, for example and without limitation, Example No. 2 illustrates formulations of storage stable pharmaceutical compositions, although one skilled in the art may provide other formulations.

In particular, in one embodiment, for example and without limitation, the specification relates to a storage stable pharmaceutical composition which comprises at least one active pharmaceutical ingredient as defined above and a pharmaceutically acceptable carrier, wherein the composition retains at least 80% of the potency of active pharmaceutical ingredient in the pharmaceutical composition after steam sterilization. In another, for example and without limitation, the specification relates to a storage stable pharmaceutical composition which comprises at least one active pharmaceutical ingredient as defined above and a pharmaceutically acceptable carrier, wherein the total amount of related substances in the composition does not increase more than 15% by weight to the active pharmaceutical ingredient after steam sterilization.

As used herein, the term “steam sterilization” refers to the process of preparing sterile pharmaceutical compositions in steam sterilizers (autoclaves), in which the sterilizing agent is water saturated steam under excessive pressure and elevated temperature. Sterilization modes are well known to specialists in this field and are for aqueous solutions and other liquid dosage forms in hermetically sealed vials, for example, 8-45 minutes at a temperature of 120-132° C. and a pressure of 120-220 kPa, and all combinations of values in between. In one embodiment, for example and without limitation, sterilization is performed for 30 minutes at a temperature of 125° C. and a pressure of 140 kPa. In another embodiment, for example and without limitation, sterilization is performed for 40 minutes at a temperature of 120° C. and a pressure of 130 kPa. In another embodiment, for example and without limitation, sterilization is performed for 20 minutes at a temperature of 132° C. and a pressure of 200 kPa.

As used herein, the term “sterile pharmaceutical composition” means a composition in which most of the microorganisms and their spores have been destroyed and which meets the sterilization criteria set forth in the US Pharmacopeia.

In particular embodiment, the pharmaceutical composition that comprises a H-Leu-D-His-Lys-Leu-Gln-Thr-NH₂ hexapeptide (SEQ ID NO: 8) and a pharmaceutically acceptable carrier, retains at least 80% of the potency of active pharmaceutical ingredient in the pharmaceutical composition and the total amount of related substances in the composition does not increase more than 15% by weight to the active pharmaceutical ingredient after steam sterilization (example No. 4) with sterilization modes 20 minutes at a temperature of 132° C. and a pressure of 220 kPa. Other sterilization modes are apparent to those skilled in the art and should be included within the scope of the present invention.

For the purposes of the present invention, the storage stable pharmaceutical composition may contain an active pharmaceutical ingredient in a concentration in the range of 1 μg/ml to 500 mg/ml. Preferably, the storage stable pharmaceutical composition of the present invention contains an active pharmaceutical ingredient in an amount sufficient to reduce the number of symptoms of pain when the pharmaceutical composition is administered to a subject in need thereof.

In a specific embodiment, Example No. 2 illustrates formulations of storage stable pharmaceutical compositions, although one skilled in the art may provide other formulations.

As used herein, the term “subject” means an animal, preferably a mammal, and most preferably a human. In the context of the present invention, the term “subject” is used interchangeably with the term “patient”. In the context of the present invention, the phrase “subject in need thereof” means a subject (patient) in need of treatment of one or more diseases, conditions or disorders (as described herein) for which the peptide drug can be suitably used.

As used herein, the term “peptide” is used in its broadest sense to mean a sequence of subunit amino acids. The peptides of the invention may contain L-amino acids, D-amino acids (which are resistant to L-amino acid-specific proteases in vivo), or a combination of D- and L-amino acids. The peptides described herein can be chemically synthesized or recombinantly expressed. The peptides can be linked to other compounds to stimulate an increased in vivo half-life, such as pegylation, HESylation, PASylation, glycosylation, or can be obtained in the form of Fc-fusion or in immunized variants. Such a bond may be covalent or non-covalent, as understood by those skilled in the art.

Peptides can also be linked to other molecules. The peptide and molecule can be linked directly to each other (for example, via a peptide bond); linked through a linker molecule, which may or may not be a peptide; or indirectly linked to each other, for example, by bonding with a common carrier molecule.

As used herein, the term “amino acid” means an organic compound containing both a basic amino group and an acidic carboxyl group. This term includes natural amino acids (e.g., L-amino acids), modified and unusual amino acids (e.g., D-amino acids), as well as amino acids that are known to occur biologically in free or combined form, but usually do not occur in proteins. This term includes modified and unusual amino acids, such as those described, for example, in [11], the description of which is incorporated herein by reference.

Natural protein amino acids include, but are not limited to, alanine, arginine, asparagine, aspartic acid, cysteine, glutamic acid, glutamine, glycine, histidine, isoleucine, leucine, lysine, methionine, phenylalanine, serine, threonine, tyrosine, tyrosine, trypto, tryptin proline and valine. Natural non-protein amino acids include, but are not limited to, arginine succinic acid, citrulline, cysteinesulfinic acid, 3,4-dihydroxyphenylalanine, homocysteine, homoserin, ornithine, 3-moniodiodyrosine, 3,5-diiodotyrosine, 3,5,5′-triiodothyronine and 3, 3′, 5.5′-tetraiodothyronine. Modified or unusual amino acids (all L-configurations except those indicated) that can be used for the practice of the invention include, but are not limited to, D-amino acids, N-methylglutamic acid (NMeGlu), D-serine (DSer), allo-threonine (AlloThr), hydroxyproline (Hyp), omithine (Orn), D-asparagine (DAsn), glutamic acid methyl ester (GluOMe), norleucine (Nie), norvaline (Nva), 1-aminocyclohexyl carboxylic acid (Chex), isoglutamine (IsoGIn), β-alanine (PAIa), 6-amino-hexanoic acid (Aha), phenylglycine, beta-phenylproline, tert-leucine, 4-aminocycle ohexylalanine, N-methylnorleucine, 3,4-dehydroproline, N, N-dimethylaminoglycine, N-methylaminoglycine, 4-aminopiperidine-4-carboxylic acid, 6 trans-4-(aminomethyl)-cyclohexanecarboxylic acid, 2-, 3- and 4-(aminomethyl) benzoic acid, 1-aminocyclopentanecarboxylic acid, 1-aminocyclopropanecarboxylic acid and 2-benzyl-5-aminopentanoic acid.

The term “unnatural amino acid” can be used to mean an amino acid that does not exist by itself in nature, but rather has been synthesized or created by man. Examples of unnatural amino acids include, but are not limited to, iodinated tyrosine, methylated tyrosine, glycosylated serine, glycosylated threonine, azetidine-2-carboxylic acid, 3,4-dehydroproline, perthiaproline, canavanin, ethionine, norleucine, selenomethionine, aminolexuric acid homoallyl glycine and homopropargylglycine.

As used herein, the term “derivative” or “modified peptide”, as used herein, refers to a peptide of five to fourteen amino acid residues containing an amino acid sequence that has at least 50% amino acid sequence identity in length relative to the amino acid sequence of peptides of the general formula 1 [SEQ ID NO: 1-144] or 2 [SEQ ID NO: 145-288], which retains peptide activity (interacting with the same specific receptor) and may differ from these sequences by truncation, deletion, substitution, addition or modification of one or more amino acids selected from a naturally occurring amino acid, a natural non-protein amino acid, a non-natural amino acid, or a modified or unusual amino acid residue, as is known to one skilled in the art.

Modified peptides may include conservative substitutions. As used herein, the term “conservative amino acid substitution” means an amino acid or nucleic acid that does not or hardly changes the peptide or polynucleotide function or other characteristics. This amino acid can be replaced by a residue having similar physicochemical characteristics, for example, by replacing one aliphatic residue with another (such as He, Val, Leu or Ala by one another) or by replacing one polar residue with another (for example, between Lys and Arg; Glu and Asp or Gin and Asn). Other such conservative substitutions, for example substitutions of entire regions having similar hydrophobicity characteristics, are well known. Peptides containing a conservative amino acid substitution can be tested in any of the assays described herein to confirm that the desired activity is maintained.

A modified peptide can also be obtained by adding one or more components at the N- and/or C-terminus of the peptide. For example, the peptides may be modified by add a label, such as FITC at the C-terminus and/or N-terminus, with or without a linker, such as aminohexanoic acid; Ahx. Peptides may also include a functional component at one or both ends of the peptide. For example, the functional component may include a targeting peptide or domain, such as an antibody or antibody fragment, a translocation peptide or domain, such as a transcriptional peptide transactivator (TAT) or a peptide or stabilization domain, with or without a linker. In particular, the N- and/or C-terminus of the modified peptides may optionally be protected from proteolysis. For example, the N-terminus may be in the form of an acetyl group, and/or the C-terminus may be in the form of an amide group.

The term “translocation peptide” or “translocation domain” refers to any amino acid sequence that directs the peptide in which it is present to a desired cellular purpose. For example, a translocation domain, such as a polyarginine sequence, can direct or facilitate the penetration of a peptide through a biological membrane, for example, a phospholipid membrane, mitochondrial membrane, or nuclear membrane. For example, the translocation sequence directs the peptide from outside the cell, through the plasma membrane, to the cytoplasm or to the desired location within the cell, for example, to the nucleus, ribosome, mitochondria, ER, lysosome or peroxisome.

Alternatively, or in addition, the translocation sequence may direct the peptide across the physiological barrier, such as the blood-brain barrier, the transmucosal barrier, or the blood-brain, hematoretinal, gastrointestinal, and pulmonary barriers.

The modified peptide can be obtained from peptides of the general formula 1 [SEQ ID NO: 1-144] or 2 [SEQ ID NO: 145-288] using any chemical modification that improves its resistance to proteolysis. For example, at least the —CONH— peptide bond can be modified and replaced by (CH2NH) reduced linkage, (NHCO) retro-inverse linkage, (CH₂—O) methyleneoxy linkage, (CH₂—S) thiomethylene linkage, (CH₂CH₂) carbamate bond, (CO—CH₂) cetomethylene bond, (CH(OH)—CH₂) hydroxyethylene bond, (N—N) bond, E-alcene bond, (—CH═CH—) bond or (CH₂SO) communication. Such substitutions and modifications are described, for example, in [12], the disclosures of which are incorporated herein by reference.

Also, a modified peptide can be obtained by acetylation, acylation, amidation, crosslinking, cyclization, disulfide bond formation, covalent crosslinking, cysteine formation, pyroglutamate formation, formylation, gamma carboxylation, glycosylation, GPI anchor formation, hydroxylation iodination, methylation, myristylation oxidation, phosphorylation and the like.

The modified peptide may be linked to other molecules. The peptide and molecule can be linked directly to each other (for example, via a peptide bond); linked through a linker molecule, which may or may not be a peptide; or indirectly linked to each other, for example, by bonding with a common carrier molecule.

The modified peptide may be cyclic or non-cyclic. Cyclic peptides in some cases have improved stability properties. Specialists in this field know how to obtain cyclic peptides.

The modified peptides described herein can be synthesized using standard synthetic methods known to those skilled in the art, for example chemical synthesis or genetic recombination. In a preferred embodiment, peptides are prepared by stepwise condensation of amino acid residues, either by condensation of a pre-formed fragment already containing an amino acid sequence in appropriate order, or by condensation of several fragments previously prepared, while protecting the amino acid functional groups except those involved in peptide bond during condensation. In particular, the peptides can be synthesized in accordance with the method originally described in [1].

In some cases, the modified peptides may include only naturally occurring amino acids, although non-natural amino acids (that is, compounds that are not found in nature but may be included in the peptide chain) that are known in the art can be used.

There are many known non-naturally occurring amino acids, any of which can be incorporated into the modified peptides of the present invention. Some examples of unnatural amino acids are 4-hydroxyproline, desmosin, gamma-aminobutyric acid, beta-cyanoalanine, norvaline, 4-(E)-butenyl-4 (R)-methyl-N-methyl-L-threonine, N-methyl-L-leucine, 1-aminocyclopropanecarboxylic acid, 1-amino-2-phenylcyclopropanecarboxylic acid, 1-aminocyclobutanecarboxylic acid, 4-aminocyclopentenecarboxylic acid, 3-aminocyclohexanecarboxylic acid, 4-piperidino-acetic acid 2,4-diaminobutyric acid, 2,3-diaminopropionic acid, 2,4-diaminobutyric acid, 2-aminoheptanedioic acid, 4-(aminomethyl) benzoic acid, 4-aminobenzoic acid, ortho-, meta- and/para-substituted phenylalanines (for example, substituted —C(═O)C₆H₅; —CF₃; —CN; -halo; —NO₂; —CH₃), disubstituted phenylalanines, substituted tyrosines (for example, additionally substituted with -Q=0)C₆H₅; —CF₃; —CN; -halo; —NO₂; —CH₃) and statin. In addition, amino acids can be derivatized to include amino acid residues that are hydroxylated, phosphorylated, sulfonated, acylated and glycosylated.

In addition, one or more amino acids in the peptide can be modified, for example, by adding a chemical element such as a carbohydrate group, a hydroxyl group, a phosphate group, a farnesyl group, an isopharnesyl group, a fatty acid group, a linker for conjugation, functionalization, or another modification, and etc. The modified peptide may also be a single molecule or may be a multimolecular complex, such as a protein. The modified peptide may be naturally occurring, recombinant or synthetic, or any combination thereof.

Examples of chemical synthesis technologies are solid phase synthesis and liquid phase synthesis. In the case of solid-phase synthesis, for example, the amino acid corresponding to the C-terminus of the peptide to be synthesized binds to a carrier that is insoluble in organic solvents, and by alternatively repeating reactions in which the amino acids with their amino groups and side chain functional groups, protected by appropriate protecting groups, condense one after the other in order from the C-terminus to the N-terminus. Solid phase synthesis methods are largely classified by the tBoc method and the Fmoc method, depending on the type of protecting group used. Commonly used protecting groups include tBoc (tert-butoxycarbonyl), CI-Z (2-chlorobenzyloxycarbonyl), Br-Z (2-bromobenzyloxycarbonyl), Bzl (benzyl), Fmoc (9-fluorenylmectoxycarbonyl), Mbh (4,4′-dimethyloxy)), Mtr (4-methoxy-2,3,6-trimethylbenzenesulfonyl), Trt (trityl), Tos (tosyl), Z (benzyloxycarbonyl) and Clz-Bzl (2,6-dichlorobenzyl) for amino groups; N02 (nitro) and Pmc (2,2,5,7,8-pentamethylchroman-6-sulfonyl) for guanidino groups); and tBu (tert-butyl) for hydroxyl groups). After synthesis of the desired peptide, it is subjected to a deprotection reaction and cut out from a solid support. Such a peptide cutting reaction can be carried out with hydrogen fluoride or trifluoromethanesulfonic acid for the Boc method and with TFA for the Fmoc method. On the other hand, the peptide can be synthesized using recombinant methods. Peptide CDK5 inhibitors can be expressed as isolated nucleic acids encoding a peptide. An isolated nucleic acid sequence may contain RNA or DNA. As used herein, “isolated nucleic acids” are those that have been removed from their normal surrounding nucleic acid sequences in the genome or in cDNA sequences. Such isolated nucleic acid sequences may contain additional sequences useful for stimulating the expression and/or purification of the encoded peptide, including, but not limited to, polyA sequences, modified Kozak sequences and sequences encoding epitope labels, export signals, and secretory signals, nuclear localization signals and plasma membrane localization signals. An isolated nucleic acid sequence may contain RNA or DNA. As used herein, “isolated nucleic acids” are those that have been removed from their normal surrounding nucleic acid sequences in the genome or in cDNA sequences.

As used herein, the term “analogue” as used in the present description refers to a molecule that is not a peptide, but similar to a peptide in at least one property, such as, for example, chain length, charge, hydrophilicity, hydrophobicity, polarity, ability to bind to hydrogen or stiffness, and also retains peptide activity (interacting with the same specific receptor).

In a specific embodiment, in the Example No. 1 are illustrated derivative (modified) peptide sequences and analogs of the developed synthetic peptides, although one skilled in the art may provide other modified peptide sequences and analogs.

As used herein, the term “pharmaceutically acceptable salt” means salt or salts of at least one of the developed synthetic peptides or their derivatives and analogues, which can be prepared by treating the developed synthetic peptides or their derivatives or their analogues with an appropriate acid or a base. Examples of pharmaceutically acceptable base addition salts include, but are not limited to, sodium, potassium, calcium, magnesium, ammonium salts or inorganic base salt. Examples of pharmaceutically acceptable organic base addition salts include, but are not limited to, those derived from organic bases such as lysine, arginine, guanidine, and the like. Examples of pharmaceutically acceptable acid addition salts include, but are not limited to, those derived from inorganic acids such as hydrochloric acid, nitric acid, phosphoric acid, sulfuric acid and the like, as well as the salts derived from organic acids such as acetic acid, trifluoroacetic acid, propionic acid, oxalic acid, maleic acid, benzoic acid, succinic acid, fumaric acid, phthalic acid, benzenesulfonic acid, p-toluenesulfonic acid, citric acid, tartaric acid, methanesulfonic acid and the like.

As used herein, the term “co-crystal” refers to a crystalline structure made up of two or more components in a definite stoichiometric ratio, where each component is defined as either an atom, ion, or molecule. The term co-crystal” encompasses within its scope many types of compounds, including hydrates, solvates and clathrates.

In a specific embodiment, in the Example No. 1 is illustrated pharmaceutically acceptable salt of the developed synthetic peptides, although one skilled in the art may provide other pharmaceutically acceptable salts and co-crystals.

For the purposes of the present invention, the pharmaceutically acceptable carrier in the storage stable pharmaceutical composition may contain a solvent selected from the group consisting of water, ethylene glycol, diethylene glycol, triethylene glycol, tetraethylene glycol, polyethylene glycol. propylene glycol, dipropylene glycol, tripropylene glycol, methoxypropylene glycol, polyethylene oxide, glycerin, ethanol, propyl alcohol, isopropyl alcohol, benzyl alcohol, benzyl benzoate, glycofurol or combinations thereof.

For the purposes of the present invention, the pharmaceutically acceptable carrier in the storage stable pharmaceutical composition may consists entirely of a solvent that is selected from the group consisting of water, ethylene glycol, diethylene glycol, triethylene glycol, tetraethylene glycol, polyethylene glycol. propylene glycol, dipropylene glycol, tripropylene glycol, methoxypropylene glycol, polyethylene oxide, glycerin, ethanol, propyl alcohol, isopropyl alcohol, benzyl alcohol, benzyl benzoate, glycofurol or combinations thereof.

For the purposes of the present invention, the pharmaceutically acceptable carrier in the storage stable pharmaceutical composition, in addition to the solvent, may contain at least one more ingredient selected from the group consisting of a pH adjusting agent; isotonicity agent; preservative; chelating agent; antioxidant; surfactant; viscosifier and absorption promoter.

As used herein, the term “pH adjusting agent” means a substance that adjusts the pH of pharmaceutical compositions to an intended pH. pH adjusting agents may include pharmaceutically acceptable acids, bases, or buffering agents. For example, acids may include, but are not limited to, one or more inorganic mineral acids such as citric, fumaric, gluconic, lactic, malic, metatartaric, tartaric, ascorbic, and benzenesulfonic acids and the like. The bases may be one or more inorganic bases or organic bases, including, but not limited to, alkaline carbonate, alkaline bicarbonate, alkaline earth metal carbonate, alkali metal hydroxide, alkaline earth metal hydroxide or amine. For example, the inorganic or organic base may be an alkaline hydroxide such as lithium hydroxide, potassium hydroxide, cesium hydroxide, sodium hydroxide or the like; alkaline carbonate such as calcium carbonate, sodium carbonate or the like; or alkaline bicarbonate such as sodium bicarbonate or the like; the organic base may also be sodium acetate. The buffering agent may be, but is not limited to, an alkali metal salt of an amino acid, aluminum hydroxide, aluminum hydroxide, magnesium glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dibasic hydrogen phosphate, dipotassium phosphate. A preferred amount of a pH adjusting agent that may be present in a shelf stable pharmaceutical composition of the present invention may be in the range of from about 0.001 to about 2.0% (mass).

As used herein, the term “pH” is a measure of the concentration of hydrogen ions that is commonly used in the art. Typically, pH provides a measure on a scale from 0 to 14 of the acidity or alkalinity of the solution. In the context of the present invention, the pH of the storage stable pharmaceutical composition of the developed synthetic peptides or their derivatives and analogues or a pharmaceutically acceptable salt and co-crystal of the above peptides, their derivatives and analogues is from about 2.0 to about 11.0, and all values and ranges in between.

As used herein, the term “isotonicity agent” as used refers to a substance in a pharmaceutical composition that serves to change the osmotic pressure of a pharmaceutical composition, such that the osmotic pressure becomes closer to that in human plasma. Examples of isotonicity agents include, but are not limited to: sodium chloride or potassium chloride or a physiologically tolerable polyol, such as, for example, sugar alcohol, in particular sorbitol or glycerin, at a concentration necessary to impart isotonicity. A preferred amount of an isotonicity agent that may be present in a shelf stable pharmaceutical composition of the present invention may be in the range of from about 0.001 to about 1.0% (mass).

As used herein, the term “preservative” refers to a substance that is added to a pharmaceutical composition to prevent or slow down microbial activity (growth and metabolism). Examples of preservatives that can be used in a shelf stable pharmaceutical composition can be selected from, but not limited to, phenylcarbinol, benzalkonium chloride, thimerosal, benzyl alcohol, ethyl alcohol, phenylethyl alcohol, methyl parabens, ethyl parabens, propyl parabens and butyl parabens, or a combination thereof. A preferred amount of preservative that may be present in the shelf stable pharmaceutical composition of the present invention may be in the range of from about 0.001 to about 2.0% (mass).

Examples of suitable chelating agents that can be used in the stable pharmaceutical composition of the present invention can be selected from, but not limited to, disodium ethylenediaminetetraacetic acid (EDTA); ethylenediaminetetraacetic acid trisodium salt; tetrasodium salt of ethylenediaminetetraacetic acid and diethyleneamine pentaacetate. A preferred amount of a chelating agent that may be present in the shelf stable pharmaceutical composition of the present invention may be in the range of from about 0.001 to about 1.0% (mass).

Examples of suitable antioxidants that can be used in the stable pharmaceutical composition of the present invention can be selected from, but not limited to, ascorbic acid, alpha-tocopherol (vitamin e), butylated hydroxyanisole, butylated hydroxytoluene, glutathione and the like. A preferred amount of an antioxidant that may be present in a shelf stable pharmaceutical composition of the present invention may range from about 0.001 to about 1.0% (mass).

Examples of suitable surfactants that can be used in the stable pharmaceutical composition of the present invention can be selected from, but not limited to polysorbates, their ether ethoxylates, produced by reaction of sorbitan esters with ethylene oxide, polyoxyethylene alkyl phenol, polyoxyethylene cetyl ether, polyoxyethylene alkyl-aryl ether, polyoxyethylene monolaurate, polyoxyethylene vegetable oil, polyoxyethylene sorbitan monolaurate, polyoxyethylene esters or mixed fatty and resin acids, polyoxyethylene sorbitol lanolin derivative, polyoxyethylene tridecylether, polyoxyethylene sorbitan esters of mixed fatty and resin acids, polyoxyethylene sorbitan monostearate, polyoxyethylene sorbitan monooleate, polyoxyethylene monostearate, polyoxyethylene stearyl ether, polyoxyethylene oleyl ether, polyoxyethylene tridecyl ether, polyoxyethylene fatty alcohol, polyoxyethylene alkyl amine, polyoxyethylene glycol monopalmitate, polyoxyethylene sorbitan monopalmitate, polyoxyethylene cetyl ether, polyoxyethylene oxypropylene stearate, polyoxyethylene lauryl ether, polyoxyethylene lanolin derivative, sodium oleate, quaternary ammonium derivative, potassium oleate, N-cetyl N-ethyl morpholinium ethosulfate, sodium lauryl sulfate or mixtures thereof. A preferred amount of surfactants that may be present in a shelf stable pharmaceutical composition of the present invention may range from about 0.001 to about 5.0% (mass).

Examples of suitable viscosifiers that can be used in the stable pharmaceutical composition of the present invention can be selected from, but not limited to, water-insoluble polymers, acrylic polymers, block copolymers of polyoxyethylene and polyoxypropylene, xanthan gum, tragacanth gum, alginates, agar agar, gelatins, sorbitol. A preferred amount of viscosifier that may be present in the shelf stable pharmaceutical composition of the present invention may be in the range of from about 0.001 to about 2.0% (mass).

Examples of suitable absorption enhancers that can be used in the stable pharmaceutical composition of the present invention can be selected from, but not limited to, disodium edetate, benzyl alcohol, ethanol, thiamine or its salt, capric acid or its salt, malic acid or its salt, pyrophosphoric acid or its salt, citric acid or its salt, salicylic acid or its salt, pyrophosphoric acid or its salt, or combinations thereof. A preferred amount of an absorption enhancer that may be present in the shelf stable pharmaceutical composition of the present invention may be in the range of from about 0.001 to about 2.0% (mass).

The invention further relates to a method of treating moderate to moderately severe pain, which requires analgesia at the opioid level in a subject in need thereof, comprising administering to the subject a storage stable pharmaceutical composition that contains at least one active pharmaceutical ingredient selected from peptides of the general formula 1 [SEQ ID NO: 1-144] or 2 [SEQ ID NO: 145-288], or their derivatives or their analogues or pharmaceutically acceptable salts and co-crystals of the above peptides, their derivatives and analogues, and a pharmaceutically acceptable carrier, wherein the composition retains at least 80% of the potency of active pharmaceutical ingredient in the pharmaceutical composition after storage for at least four months at room temperature.

The specification further relates to a process of preparing a storage stable pharmaceutical composition, the process comprising, but not limited to, a steam sterilization stage that provides improved stability.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1. Synthesis of Developed Synthetic Peptide and their Derivatives, Analogues and Salts

The H-Leu-D-His-Lys-Leu-Gln-Thr-NH₂ hexapeptide [SEQ ID NO: 8] synthesis was carried out by automatic solid-phase Fmoc synthesis on the Rink polymer (Rink Amide Resin, 0.6 mmol of amino-groups per 1 g of polymer) using the DCC/HOBt (N,N′-dicyclohexylcarbodiimide/1-hydroxybenzotriazole) amino-acid activation method. Deblocking was carried out by treatment with piperidine/DMF (piperidine/N,N-dimethylformamide) (1:4) solution during 7 minutes. The side chain groups were protected by the following groups: tBu (tert-butyl ether) for tyrosine, threonine, Trt (trityl or triphenylmethyl) for glutamine and histidine, Boc (t-butyloxycarbonyl) for lysine. The peptides were removed from the polymer and deblocked by the TFA/H2O/EDT (trifluoroacetic acid/water/1,2-ethanediol) (90:5:5) mixture. The peptide was purified by reverse-phase HPLC (C18 column), eluent-acetonitrile-water (0.1 M potassium dihydrophosphate) at a ratio of 6:4.

The peptide acetate was obtained by anion exchange on the same reverse phase HPLC on which the peptide was purified. The peptide is loaded onto a column, washed with a sufficient amount of acetic acid buffer, and then eluted using an aqueous acetic acid/acetonitrile gradient. After freeze-drying, the TFA counterion will be replaced. The technique is based on the hydrophobicity of the peptide, and a very hydrophilic peptide will require a corresponding anion exchange resin.

The peptide was described by mass spectrometer and HPLC, Waters DeltaPak C18 3.9*150 mm 5u 100 A column; solution A: 0.1% TFA in 100% water/MeCN; with a flow rate of 1 ml/min; detection wavelength of 230 nm.

The amount of pharmaceutically active ingredient in the pharmaceutical composition was also determined by HPLC in accordance with the method described in [13].

Using the same technology, were synthesized:

1. Derivative (modified) peptide sequence of the developed synthetic peptide: H-Leu-D-His-Lys-Leu-Gln-Thr-CON₂H₂(OMe)₂ (the modified peptide is obtained by substitution one components at the C-terminus of the peptide [SEQ ID NO: 7] with dimethyl hydrazide);

2. Derivative (modified) peptide sequence of the developed synthetic peptide: Palmitoyl-Leu-D-His-Lys-Leu-Gln-Thr-NH₂ (the modified peptide is obtained by addition with Palmitoyl at the N-terminus of the peptide [SEQ ID NO: 8]);

3. Derivative (modified) peptide sequence of the developed synthetic peptide: H-His-Lys-Leu-Gln-Thr-OMe (the modified peptide is obtained by deletion one components at the N-terminus of the peptide [SEQ ID NO: 1]);

4. Analogue of the developed synthetic peptide: H-Leu-His-Lys-(BOC)-Leu-Gln-Thr-Phe-Pro-Arg-OH, wherein (BOC)=(CH₃)₃C—O—C(═O)— (the peptide analog is obtained from peptide [SEQ ID NO: 2] by addition with (BOC) and modification of the C-terminus of the peptide);

5. Salt (acetate) of the peptide [SEQ ID NO: 8]: H-Leu-D-His-Lys-Leu-Gln-Thr-NH₂.(CH₃COOH);

6. The H-Leu-D-His-Lys-Leu-Gln-Thr-OMe hexapeptide [SEQ ID NO: 7].

### Example 2. Preparing a Storage Stable Pharmaceutical Composition

The preparation of the pharmaceutical composition for the purposes of the present invention included the following stages:

(a) the preparation of a pharmaceutically acceptable carrier,

(b) adding to the carrier an active pharmaceutical ingredient synthesized in accordance with Example 1 with continuous stirring;

(c) optionally adding at least one more ingredient selected from the group consisting of a pH adjusting agent; isotonicity agent; preservative; chelating agent; antioxidant; surfactant; viscosifier or absorption promoter (for pharmaceutical compositions No. 1, 2, 4, 5, 6, 7, 8, 9);

(d) filling the pharmaceutical composition in 1 ml glass vials followed by closing;

(e) optionally steam sterilization (for pharmaceutical compositions No. 1, 3, 4, 5, 6, 7).

The following pharmaceutical compositions were prepared:

TABLE 1
Storage stable pharmaceutical compositions

| Pharmaceutical | | Quantity of | Pharmaceutical |
|---|---|---|---|
| composition No. | Ingredients | Ingredients % w/w | composition pH |
| 1. Steam sterilized | Peptide [SEQ ID NO: 8] | 0.10 | 5.0 ± 0.1 |
| | Hydrochloric acid | q.s. to pH | |
| | | 5.0 ± 0.1 | |
| | Purified Water | q.s. to 100% | |
| 2. | Peptide [SEQ ID NO: 8] | 0.80 | 3.7 ± 0.1 |
| | Acetic acid | 0.22 | |
| | Sodium acetate trihydrate | 0.20 | |
| | Benzalkonium chloride | 0.01 | |
| | Sodium chloride | 0.50 | |
| | EDTA | 0.005 | |
| | Ascorbic acid | 0.02 | |
| | Polysorbate TWEEN 20 | 0.01 | |
| | Gelatin | 1.00 | |
| | Purified Water | q.s. to 100% | |
| 3. Steam sterilized | Peptide [SEQ ID NO: 8] | 0.10 | 7.0 ± 0.1 |
| | Polypropylene glycol/glycerin | q.s. to 100% | |
| | mixture (50/50) | | |
| 4. Steam sterilized | Salt (acetate) of the peptide | 0.10 | 4.0 ± 0.1 |
| | [SEQ ID NO: 8] | | |
| | Sodium chloride | 0.50 | |
| | Hydrochloric acid | q.s. to pH | |
| | | 4.0 ± 0.1 | |
| | Purified Water | q.s. to 100% | |
| 5. Steam sterilized | Derivative ( modified ) peptide | 0.10 | 4.0 ± 0.1 |
| | No. 1 | | |
| | Sodium chloride | 0.50 | |
| | Hydrochloric acid | q.s. to pH | |
| | | 4.0 ± 0.1 | |
| | Purified Water | q.s. to 100% | |
| 6. Steam sterilized | Derivative (modified) peptide No. 2 | 0.10 | 4.0 ± 0.1 |
| | Sodium chloride | 0.50 | |
| | Hydrochloric acid | q.s. to pH | |
| | | 4.0 ± 0.1 | |
| | Purified Water | q.s. to 100% | |
| 7. Steam sterilized | Peptide [SEQ ID NO: 7] | 0.12 | 4.0 ± 0.1 |
| | Sodium chloride | 0.50 | |
| | Hydrochloric acid | q.s. to pH | |
| | | 4.0 ± 0.1 | |
| | Purified Water | q.s. to 100% | |
| 8. | Derivative (modified) peptide | 0.12 | 4.0 ± 0.1 |
| | No. 3 | | |
| | Benzalkonium chloride | 0.01 | |
| | Sodium chloride | 0.50 | |
| | Hydrochloric acid | q.s. to pH | |
| | | 4.0 ± 0.1 | |
| | Purified Water | q.s. to 100% | |
| 9. | Analogue of the developed | 0.12 | 4.0 ± 0.1 |
| | synthetic peptide | | |
| | Benzalkonium chloride | 0.01 | |
| | Sodium chloride | 0.50 | |
| | Hydrochloric acid | q.s. to pH | |
| | | 4.0 ± 0.1 | |
| | Purified Water | q.s. to 100% | |

### Example 3. The Study of Storage Stability of the Pharmaceutical

The study of storage stability of pharmaceutical compositions for the purposes of the present invention was carried out as follows:

1. The pharmaceutical compositions No. 1 and 2 from the Table 1 of the present invention in closed vials were stored at room temperature 25° C. for four months. As a control, a composition, similar to pharmaceutical composition No. 1, but without special measures by doing stability, in closed vials was stored at room temperature 25° C. for four months.

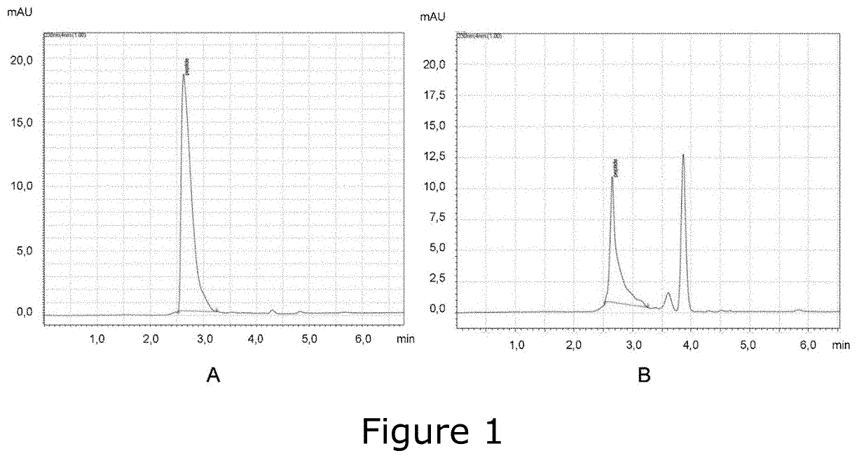

2. The amount of active pharmaceutical ingredient and related substances was determined using HPLC. The results of the study are shown in FIGS. 1, 2 and 3.

FIG. 1 is a chromatogram of a composition, similar to pharmaceutical composition No. 1, but without special measures by doing stability, after preparation (A) and after four months of storage at room temperature 25° C. (B). The amount of active pharmaceutical ingredient after four months of storage was 50.1% of the initial value, the amount of related impurities exceed 49%.

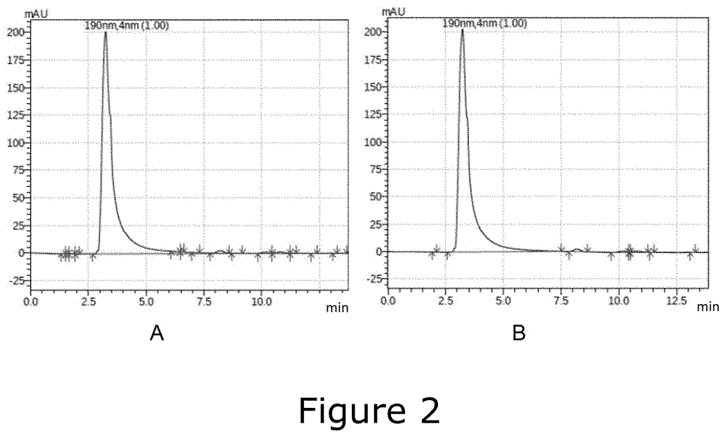

FIG. 2 is a chromatogram of a steam sterilized pharmaceutical composition No. 1 after preparation (A) and after four months of storage at room temperature 25° C. (B). The amount of active pharmaceutical ingredient after four months of storage was 98.3% of the initial value, the amount of related impurities did not exceed 1.5%.

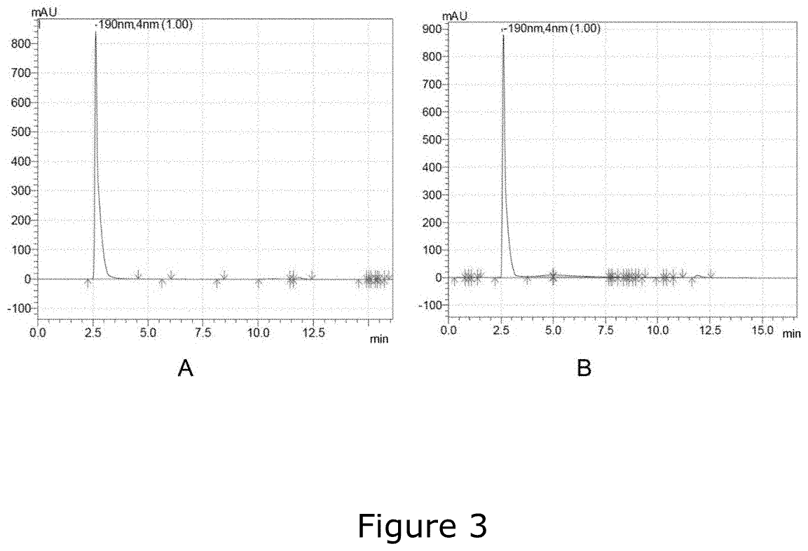

FIG. 3 is a chromatogram of sterilized pharmaceutical composition No. 2 after preparation (A) and after four months of storage at room temperature 25° C. (B). The amount of active pharmaceutical ingredient after four months of storage was 97.7% of the initial value, the amount of related impurities did not exceed 2.0%.

The result indicates that the compositions of the present invention remain stable and retain the potential of the active pharmaceutical ingredient over a storage period of at least 80%.

The results also showed that the compositions of the present invention have excellent storage stability and do not require a cold chain with a temperature range of 2° C. to 8° C.

### Example 4. The Study of Stability after Steam Sterilization of the Pharmaceutical Compositions

The study of stability after steam sterilization of pharmaceutical compositions for the purposes of the present invention was carried out as follows:

1. The pharmaceutical composition No. 3 from the Table 1 of the present invention in closed vials was subjected to steam sterilization with sterilization modes 20 minutes at a temperature of 132° C. and a pressure of 220 kPa.

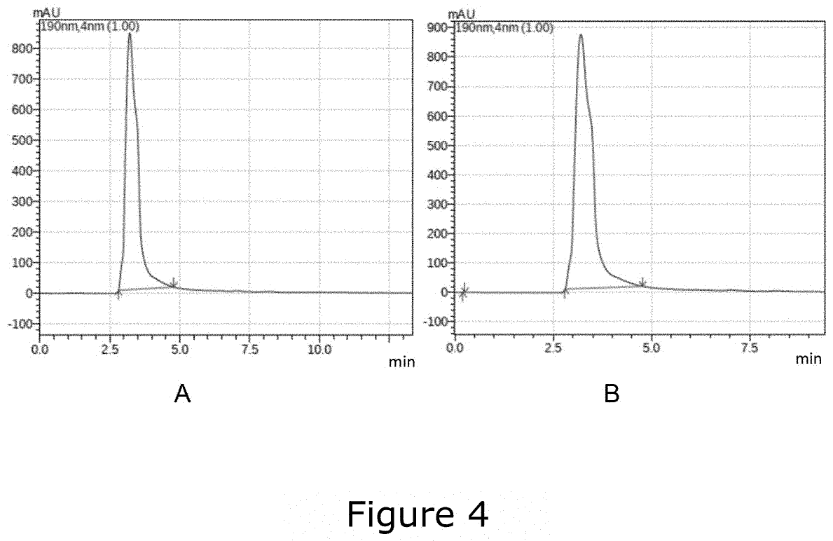

2. The amount of active pharmaceutical ingredient and related substances was determined using HPLC. The results of the study are shown in FIG. 4.

FIG. 4 is a chromatogram of pharmaceutical composition No. 3 after preparation (A) and after steam sterilization (B). The amount of active pharmaceutical ingredient after steam sterilization was 97.1% of the initial value, the amount of related impurities did not exceed 2.7%.

The result indicates that the compositions of the present invention remain stable and retain the potential of the active pharmaceutical ingredient after steam sterilization of at least 80%. Alternatively, the total amount of related substances in the composition does not increase more than 15% by weight to the active pharmaceutical ingredient after steam sterilization.

### Example 5. The Study of Analgesic Activity of the Pharmaceutical Compositions

The “Formalin Test” [14] and “Tooth pulp electro-stimulation Test” [15] were selected as pain models for the purpose of studying the analgesic activity of the pharmaceutical compositions of the present invention.

Purposefully bred and previously not involved in the study animals were used in the experiments. The animals were kept in premises with controlled conditions at an air temperature of 20-26° C. and a relative humidity of 30-70%, with a regular change of the light cycle (12-hours light/12-hours darkness) with water and food ad libitum. All animals underwent the 30-day quarantine before being sent and the 7-day adaptation after being delivered to vivaria. During the adaptation period, the clinical health signs (overall health, liveliness, cleanliness, fatness, appetite, absence of symptoms) were visually controlled in animals. Before beginning the study, the animals that met the criteria for inclusion in the experiment (species, gender, body weight, clinical health) were distributed into the appropriate groups. The distribution was made so that the average weights of animal groups of a single species differed by no more than 10%. Each animal was assigned with a unique number.

The animal experiments were carried out in compliance with the legal and ethical standards for animal treatment in accordance with the regulations adopted by the European Convention for the Protection of Vertebrate Animals Used for Experimental and Other Scientific Purposes (European Convention for the Protection of Vertebrate Animals Used for Experimental and other Scientific Purposes (ETS 123). Strasbourg, 1986).

Formalin test. The Formalin Test was carried out in the following modification: 20 minutes before the formalin administration into the paw, the outbred male rats aged about 3 months weighing 180-200 g were subocciptally under light ether anesthesia injected 10 μl with prepared stable pharmaceutical compositions or the saline solution for the control group. Rats were administered an aqueous formalin solution in a 1:50 dilution in a volume of 50 μl subplantarly (under plantar aponeurosis) of the right hind paw. After administration of formalin solution, an animal was immediately placed in a transparent cylinder to register pain reactions. All animal experiments were performed in double-blind in accordance with GLP principles.

The initial time of the first phase of the pain reaction, duration of the first phase of the pain reaction, initial time of the second phase of the pain reaction and the second pain phase presence were estimated.

TABLE 2
The result of a study of the analgesic activity of pharmaceutical
compositions (Formalin Test)

| | | Initial time of | Duration of | Initial time of | Number of |
|---|---|---|---|---|---|
| | | the first | the first | the second | rats without |
| | | phase of the | phase of the | phase of the | second |
| Pharmaceutical | | pain reaction | pain reaction | pain reaction | phase of the |
| composition No. | Number of animals | (sec) | (sec) | (min) | pain reaction |
| Control group | 22 | 6 ± 2 | 484 ± 17 | 17 ± 8 | — |
| 4 | 15 | 56 ± 17 | 296 ± 41 | 18 ± 2 | 7 |
| | | p <0.01 | p <0.01 | n = 8 | |
| 5 | 8 | 60 ± 9 | 182 ± 23 | 17 ± 2 | 3 |
| | | p <0.01 | p <0.001 | n = 5 | |
| 6 | 5 | 18 ± 11 | 394 ± 40 | 19 ± 5 | 2 |
| | | p <0.01 | p <0.01 | n = 3 | |

The result indicates that the storage stable pharmaceutical compositions of the present invention have pronounced analgesic activity.

Tooth pulp electro-stimulation Test. The Tooth pulp electro-stimulation Test for cats was carried out in the following modification: previously, under nembutal anesthesia, cavities were formed in animals in the dentine of the canine and molar of one of the sides of the upper jaw without opening the pulp chamber. A filling material, a silver amalgam, was introduced into the cavity, into which was placed the tin-coated end of a stranded copper wire in teflon insulation. The tooth was closed with quick-hardening filling material—acrylic oxide. The other end of the wire through a thin steel cannula was performed subcutaneously with led out through the skin in the occipital region of the animal. An indifferent electrode was inserted through the skin incision into the bones of the frontal sinus. The experiments were started 5-7 days after the operation of implantation of the electrodes and after the animals were completely restored after the operation. Electric stimulation of the teeth was carried out with the EOM-3 device, which is widely used in the clinic of therapeutic dentistry to determine the thresholds of pain sensitivity in patients.

The analgesic activity of the drugs was evaluated based on a change in the painful behavioral response in response to electrical stimulation of the tooth pulp before and after drug administration. In a painful behavioral reaction, three levels were distinguished with corresponding thresholds for the excitation of pain receptors, developing sequentially with increasing intensity of electrical stimulation:
The first level was characterized by a manifestation of the reaction of opening the mouth;
The second level was characterized by the appearance of the motor reaction of the animal, which consists in turning and tilting the head with its mouth wide open;
The third level was manifested by a pronounced defensive reaction, in an attempt to escape and a voice reaction.

The threshold values for the excitation of painful pulp receptors in the tooth, leading to the corresponding level of behavioral reaction, were determined at interval 60 minutes after the administration of the drugs by two independent observers. All animal experiments were performed in double blind mode testing in accordance with the principles of GLP.

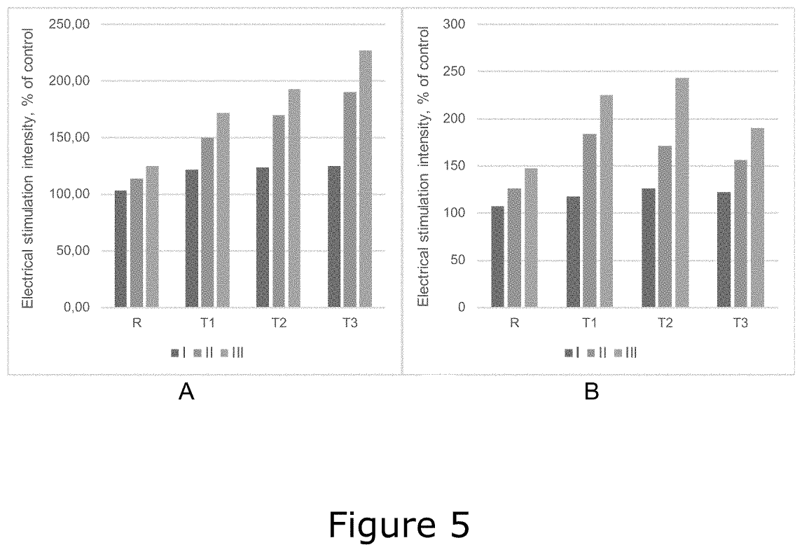

Prepared stable pharmaceutical compositions, comparison drug (tramadol hydrochloride), and control (physiological saline) were administered suboccipitally under light ether anesthesia. After 15-20 minutes in animals, the external signs of ether anesthesia has completely disappeared. The results of the study are shown in FIG. 5.

FIG. 5. The values of the excitation threshold of painful receptors for tooth pulp in animals, leading to the corresponding level of behavioral reaction, as a percentage of control 60 minutes after suboccipital administration of tramadol hydrochloride at a dose of 12 mg/kg (R), pharmaceutical composition 7 at a dose of 180 μg/kg (T1), pharmaceutical composition 8 at a dose of 180 μg/kg (T2), pharmaceutical composition 9 at a dose of 180 μg/kg (T3), for the canine (A) and molar (B), respectively (I; II; III—levels of manifestation of pain reaction).

The result indicates that the storage stable pharmaceutical compositions of the present invention have pronounced analgesic activity at the opioid level.

### REFERENCES

1. Vlasov, G. P., Kotin, A. M. (2012). Synthetic peptides with a non-narcotic type of analgesic effect. U.S. Pat. No. 9,260,482 Washington, D.C.: U.S. Patent and Trademark Office.
2. Kotin, A. M., Emelyanov, M. O., Kotin, O. A. (2017). Specific peptide clathrin inhibitors. PCT No. WO2018186770. World Intellectual Property Organization, International Bureau.
3. Kotin, A. M., Emelyanov, M. O., Kotin, O. A. (2019). Low-molecular synthetic peptides with non-narcotic type of analgesia: comparative study and mechanism of analgesic activity. Molecular pain, 15: 1-13.
4. Juul-Mortensen, C., Engelund, D. K. (2009). U.S. Pat. No. 7,632,806. Washington, D.C.: U.S. Patent and Trademark Office.
5. Stem, W. (2002). U.S. Pat. No. 6,440,392. Washington, D.C.: U.S. Patent and Trademark Office.
6. Sonavaria, V., Updhyay, K. K., & Patel, P. (2016). PCT No. WO2016059592. World Intellectual Property Organization, International Bureau.
7. Usach, I., Martinez, R., Festini, T., & Peris, J. E. (2019). Subcutaneous Injection of Drugs: Literature Review of Factors Influencing Pain Sensation at the Injection Site. Advances in therapy, 1-11.
8. Defelippis, M. R., Dobbins, M. A., Sharknas, A. D., Prokai, A. M., Rinella, J. V. (2006). U.S. Pat. No. 7,022,674. Washington, D.C.: U.S. Patent and Trademark Office.
9. Cynthia L. S., Sally A. T., Steven J. P., Jeremy C. W., Joe L., James B. E. (1996). Patent No. 2259403. Canadian Intellectual Property Office.
10. Guideline. (2003). Stability testing of new drug substances and products. ICH Topic Q1A (R2), 4, 1-24.
11. Roberts, D. C. and Vellaccio, F. (1983). The Peptides. Gross, E.; Meienhofer, J., Eds.; Academic Press: New York, N.Y.
12. Fauchere, J. L. (1986). Advances in Drug Research. B Tesla ed., vol. 15, Academic Press, London.
13. Meyer, V. R. (1992). Use of chromatographic peak-heights ratios for quantitative analysis: application to the separation of enantiomers. Journal of Chromatography A, 623(2), 371-374.
14. Tjølsen A, Berge O-G, Hunskaar S, Rosland J H and Hole K. (1992) The formalin test: an evaluation of the method. Pain, 51(1), 5-17.
15. Le Bars D, Gozariu M, Cadden S W. (2001) Animal Models of Nociception. Pharmacological Reviews, 53, 597-652.

Certain adaptations and modifications of the described embodiments can be made. Therefore, the above discussed embodiments are considered to be illustrative and not restrictive.

List of peptides of the general formula 1 [SEQ ID NO: 1-144] and
2 [SEQ ID NO: 145-288]

| SEQ ID NO | Sequence |
|---|---|
| 1 | L-Leu- L-His- L-Lys- L-Leu- L-Gln- L-Thr- OMe |
| 2 | L-Leu- L-His- L-Lys- L-Leu- L-Gln- L-Thr- NH₂ |
| 3 | L-Ala- L-His- L-Lys- L-Leu- L-Gln- L-Thr- OMe |
| 4 | L-Ala- L-His- L-Lys- L-Leu- L-Gln- L-Thr- NH₂ |
| 5 | D-Ala- L-His- L-Lys- L-Leu- L-Gln- L-Thr- OMe |
| 6 | D-Ala- L-His- L-Lys- L-Leu- L-Gln- L-Thr- NH₂ |
| 7 | L-Leu- D-His- L-Lys- L-Leu- L-Gln- L-Thr- OMe |
| 8 | L-Leu- D-His- L-Lys- L-Leu- L-Gln- L-Thr- NH₂ |
| 9 | L-Ala- D-His- L-Lys- L-Leu- L-Gln- L-Thr- OMe |
| 10 | L-Ala- D-His- L-Lys- L-Leu- L-Gln- L-Thr- NH₂ |
| 11 | D-Ala- D-His- L-Lys- L-Leu- L-Gln- L-Thr- OMe |
| 12 | D-Ala- D-His- L-Lys- L-Leu- L-Gln- L-Thr- NH₂ |
| 13 | L-Leu- L-Ala- L-Lys- L-Leu- L-Gln- L-Thr- OMe |
| 14 | L-Leu- L-Ala- L-Lys- L-Leu- L-Gln- L-Thr- NH₂ |
| 15 | L-Ala- L-Ala- L-Lys- L-Leu- L-Gln- L-Thr- OMe |
| 16 | L-Ala- L-Ala- L-Lys- L-Leu- L-Gln- L-Thr- NH₂ |
| 17 | D-Ala- L-Ala- L-Lys- L-Leu- L-Gln- L-Thr- OMe |
| 18 | D-Ala- L-Ala- L-Lys- L-Leu- L-Gln- L-Thr- NH₂ |
| 19 | L-Leu- D-Ala- L-Lys- L-Leu- L-Gln- L-Thr- OMe |
| 20 | L-Leu- D-Ala- L-Lys- L-Leu- L-Gln- L-Thr- NH₂ |
| 21 | L-Ala- D-Ala- L-Lys- L-Leu- L-Gln- L-Thr- OMe |
| 22 | L-Ala- D-Ala- L-Lys- L-Leu- L-Gln- L-Thr- NH₂ |
| 23 | D-Ala- D-Ala- L-Lys- L-Leu- L-Gln- L-Thr- OMe |
| 24 | D-Ala- D-Ala- L-Lys- L-Leu- L-Gln- L-Thr- NH₂ |
| 25 | L-Leu- L-His- L-Lys- L-Leu- L-Ala- L-Thr- OMe |
| 26 | L-Leu- L-His- L-Lys- L-Leu- L-Ala- L-Thr- NH₂ |
| 27 | L-Ala- L-His- L-Lys- L-Leu- L-Ala- L-Thr- OMe |
| 28 | L-Ala- L-His- L-Lys- L-Leu- L-Ala- L-Thr- NH₂ |
| 29 | D-Ala- L-His- L-Lys- L-Leu- L-Ala- L-Thr- OMe |
| 30 | D-Ala- L-His- L-Lys- L-Leu- L-Ala- L-Thr- NH₂ |
| 31 | L-Leu- D-His- L-Lys- L-Leu- L-Ala- L-Thr- OMe |
| 32 | L-Leu- D-His- L-Lys- L-Leu- L-Ala- L-Thr- NH₂ |
| 33 | L-Ala- D-His- L-Lys- L-Leu- L-Ala- L-Thr- OMe |
| 34 | L-Ala- D-His- L-Lys- L-Leu- L-Ala- L-Thr- NH₂ |
| 35 | D-Ala- D-His- L-Lys- L-Leu- L-Ala- L-Thr- OMe |
| 36 | D-Ala- D-His- L-Lys- L-Leu- L-Ala- L-Thr- NH₂ |
| 37 | L-Leu- L-Ala- L-Lys- L-Leu- L-Ala- L-Thr- OMe |
| 38 | L-Leu- L-Ala- L-Lys- L-Leu- L-Ala- L-Thr- NH₂ |
| 39 | L-Ala- L-Ala- L-Lys- L-Leu- L-Ala- L-Thr- OMe |
| 40 | L-Ala- L-Ala- L-Lys- L-Leu- L-Ala- L-Thr- NH₂ |
| 41 | D-Ala- L-Ala- L-Lys- L-Leu- L-Ala- L-Thr- OMe |
| 42 | D-Ala- L-Ala- L-Lys- L-Leu- L-Ala- L-Thr- NH₂ |
| 43 | L-Leu- D-Ala- L-Lys- L-Leu- L-Ala- L-Thr- OMe |
| 44 | L-Leu- D-Ala- L-Lys- L-Leu- L-Ala- L-Thr- NH₂ |
| 45 | L-Ala- D-Ala- L-Lys- L-Leu- L-Ala- L-Thr- OMe |
| 46 | L-Ala- D-Ala- L-Lys- L-Leu- L-Ala- L-Thr- NH₂ |
| 47 | D-Ala- D-Ala- L-Lys- L-Leu- L-Ala- L-Thr- OMe |
| 48 | D-Ala- D-Ala- L-Lys- L-Leu- L-Ala- L-Thr- NH₂ |
| 49 | L-Leu- L-His- L-Lys- L-Leu- D-Ala- L-Thr- OMe |
| 50 | L-Leu- L-His- L-Lys- L-Leu- D-Ala- L-Thr- NH₂ |
| 51 | L-Ala- L-His- L-Lys- L-Leu- D-Ala- L-Thr- OMe |
| 52 | L-Ala- L-His- L-Lys- L-Leu- D-Ala- L-Thr- NH₂ |
| 53 | D-Ala- L-His- L-Lys- L-Leu- D-Ala- L-Thr- OMe |
| 54 | D-Ala- L-His- L-Lys- L-Leu- D-Ala- L-Thr- NH₂ |
| 55 | L-Leu- D-His- L-Lys- L-Leu- D-Ala- L-Thr- OMe |
| 56 | L-Leu- D-His- L-Lys- L-Leu- D-Ala- L-Thr- NH₂ |
| 57 | L-Ala- D-His- L-Lys- L-Leu- D-Ala- L-Thr- OMe |
| 58 | L-Ala- D-His- L-Lys- L-Leu- D-Ala- L-Thr- NH₂ |
| 59 | D-Ala- D-His- L-Lys- L-Leu- D-Ala- L-Thr- OMe |
| 60 | D-Ala- D-His- L-Lys- L-Leu- D-Ala- L-Thr- NH₂ |
| 61 | L-Leu- L-Ala- L-Lys- L-Leu- D-Ala- L-Thr- OMe |
| 62 | L-Leu- L-Ala- L-Lys- L-Leu- D-Ala- L-Thr- NH₂ |
| 63 | L-Ala- L-Ala- L-Lys- L-Leu- D-Ala- L-Thr- OMe |
| 64 | L-Ala- L-Ala- L-Lys- L-Leu- D-Ala- L-Thr- NH₂ |
| 65 | D-Ala- L-Ala- L-Lys- L-Leu- D-Ala- L-Thr- OMe |
| 66 | D-Ala- L-Ala- L-Lys- L-Leu- D-Ala- L-Thr- NH₂ |
| 67 | L-Leu- D-Ala- L-Lys- L-Leu- D-Ala- L-Thr- OMe |
| 68 | L-Leu- D-Ala- L-Lys- L-Leu- D-Ala- L-Thr- NH₂ |
| 69 | L-Ala- D-Ala- L-Lys- L-Leu- D-Ala- L-Thr- OMe |
| 70 | L-Ala- D-Ala- L-Lys- L-Leu- D-Ala- L-Thr- NH₂ |
| 71 | D-Ala- D-Ala- L-Lys- L-Leu- D-Ala- L-Thr- OMe |
| 72 | D-Ala- D-Ala- L-Lys- L-Leu- D-Ala- L-Thr- NH₂ |
| 73 | L-Tyr- L-Leu- L-His- L-Lys- L-Leu- L-Gln- L-Thr- OMe |
| 74 | L-Tyr- L-Leu- L-His- L-Lys- L-Leu- L-Gln- L-Thr- NH₂ |
| 75 | L-Tyr- L-Ala- L-His- L-Lys- L-Leu- L-Gln- L-Thr- OMe |
| 76 | L-Tyr- L-Ala- L-His- L-Lys- L-Leu- L-Gln- L-Thr- NH₂ |
| 77 | L-Tyr- D-Ala- L-His- L-Lys- L-Leu- L-Gln- L-Thr- OMe |
| 78 | L-Tyr- D-Ala- L-His- L-Lys- L-Leu- L-Gln- L-Thr- NH₂ |
| 79 | L-Tyr- L-Leu- D-His- L-Lys- L-Leu- L-Gln- L-Thr- OMe |
| 80 | L-Tyr- L-Leu- D-His- L-Lys- L-Leu- L-Gln- L-Thr- NH₂ |
| 81 | L-Tyr- L-Ala- D-His- L-Lys- L-Leu- L-Gln- L-Thr- OMe |
| 82 | L-Tyr- L-Ala- D-His- L-Lys- L-Leu- L-Gln- L-Thr- NH₂ |
| 83 | L-Tyr- D-Ala- D-His- L-Lys- L-Leu- L-Gln- L-Thr- OMe |
| 84 | L-Tyr- D-Ala- D-His- L-Lys- L-Leu- L-Gln- L-Thr- NH₂ |
| 85 | L-Tyr- L-Leu- L-Ala- L-Lys- L-Leu- L-Gln- L-Thr- OMe |
| 86 | L-Tyr- L-Leu- L-Ala- L-Lys- L-Leu- L-Gln- L-Thr- NH₂ |
| 87 | L-Tyr- L-Ala- L-Ala- L-Lys- L-Leu- L-Gln- L-Thr- OMe |
| 88 | L-Tyr- L-Ala- L-Ala- L-Lys- L-Leu- L-Gln- L-Thr- NH₂ |
| 89 | L-Tyr- D-Ala- L-Ala- L-Lys- L-Leu- L-Gln- L-Thr- OMe |
| 90 | L-Tyr- D-Ala- L-Ala- L-Lys- L-Leu- L-Gln- L-Thr- NH₂ |
| 91 | L-Tyr- L-Leu- D-Ala- L-Lys- L-Leu- L-Gln- L-Thr- OMe |
| 92 | L-Tyr- L-Leu- D-Ala- L-Lys- L-Leu- L-Gln- L-Thr- NH₂ |
| 93 | L-Tyr- L-Ala- D-Ala- L-Lys- L-Leu- L-Gln- L-Thr- OMe |
| 94 | L-Tyr- L-Ala- D-Ala- L-Lys- L-Leu- L-Gln- L-Thr- NH₂ |
| 95 | L-Tyr- D-Ala- D-Ala- L-Lys- L-Leu- L-Gln- L-Thr- OMe |
| 96 | L-Tyr- D-Ala- D-Ala- L-Lys- L-Leu- L-Gln- L-Thr- NH₂ |
| 97 | L-Tyr- L-Leu- L-His- L-Lys- L-Leu- L-Ala- L-Thr- OMe |
| 98 | L-Tyr- L-Leu- L-His- L-Lys- L-Leu- L-Ala- L-Thr- NH₂ |
| 99 | L-Tyr- L-Ala- L-His- L-Lys- L-Leu- L-Ala- L-Thr- OMe |
| 100 | L-Tyr- L-Ala- L-His- L-Lys- L-Leu- L-Ala- L-Thr- NH₂ |
| 101 | L-Tyr- D-Ala- L-His- L-Lys- L-Leu- L-Ala- L-Thr- OMe |
| 102 | L-Tyr- D-Ala- L-His- L-Lys- L-Leu- L-Ala- L-Thr- NH₂ |
| 103 | L-Tyr- L-Leu- D-His- L-Lys- L-Leu- L-Ala- L-Thr- OMe |
| 104 | L-Tyr- L-Leu- D-His- L-Lys- L-Leu- L-Ala- L-Thr- NH₂ |
| 105 | L-Tyr- L-Ala- D-His- L-Lys- L-Leu- L-Ala- L-Thr- OMe |
| 106 | L-Tyr- L-Ala- D-His- L-Lys- L-Leu- L-Ala- L-Thr- NH₂ |
| 107 | L-Tyr- D-Ala- D-His- L-Lys- L-Leu- L-Ala- L-Thr- OMe |
| 108 | L-Tyr- D-Ala- D-His- L-Lys- L-Leu- L-Ala- L-Thr- NH₂ |
| 109 | L-Tyr- L-Leu- L-Ala- L-Lys- L-Leu- L-Ala- L-Thr- OMe |
| 110 | L-Tyr- L-Leu- L-Ala- L-Lys- L-Leu- L-Ala- L-Thr- NH₂ |
| 111 | L-Tyr- L-Ala- L-Ala- L-Lys- L-Leu- L-Ala- L-Thr- OMe |
| 112 | L-Tyr- L-Ala- L-Ala- L-Lys- L-Leu- L-Ala- L-Thr- NH₂ |
| 113 | L-Tyr- D-Ala- L-Ala- L-Lys- L-Leu- L-Ala- L-Thr- OMe |
| 114 | L-Tyr- D-Ala- L-Ala- L-Lys- L-Leu- L-Ala- L-Thr- NH₂ |
| 115 | L-Tyr- L-Leu- D-Ala- L-Lys- L-Leu- L-Ala- L-Thr- OMe |
| 116 | L-Tyr- L-Leu- D-Ala- L-Lys- L-Leu- L-Ala- L-Thr- NH₂ |
| 117 | L-Tyr- L-Ala- D-Ala- L-Lys- L-Leu- L-Ala- L-Thr- OMe |
| 118 | L-Tyr- L-Ala- D-Ala- L-Lys- L-Leu- L-Ala- L-Thr- NH₂ |
| 119 | L-Tyr- D-Ala- D-Ala- L-Lys- L-Leu- L-Ala- L-Thr- OMe |
| 120 | L-Tyr- D-Ala- D-Ala- L-Lys- L-Leu- L-Ala- L-Thr- NH₂ |
| 121 | L-Tyr- L-Leu- L-His- L-Lys- L-Leu- D-Ala- L-Thr- OMe |
| 122 | L-Tyr- L-Leu- L-His- L-Lys- L-Leu- D-Ala- L-Thr- NH₂ |
| 123 | L-Tyr- L-Ala- L-His- L-Lys- L-Leu- D-Ala- L-Thr- OMe |
| 124 | L-Tyr- L-Ala- L-His- L-Lys- L-Leu- D-Ala- L-Thr- NH₂ |
| 125 | L-Tyr- D-Ala- L-His- L-Lys- L-Leu- D-Ala- L-Thr- OMe |
| 126 | L-Tyr- D-Ala- L-His- L-Lys- L-Leu- D-Ala- L-Thr- NH₂ |
| 127 | L-Tyr- L-Leu- D-His- L-Lys- L-Leu- D-Ala- L-Thr- OMe |
| 128 | L-Tyr- L-Leu- D-His- L-Lys- L-Leu- D-Ala- L-Thr- NH₂ |
| 129 | L-Tyr- L-Ala- D-His- L-Lys- L-Leu- D-Ala- L-Thr- OMe |
| 130 | L-Tyr- L-Ala- D-His- L-Lys- L-Leu- D-Ala- L-Thr- NH₂ |
| 131 | L-Tyr- D-Ala- D-His- L-Lys- L-Leu- D-Ala- L-Thr- OMe |
| 132 | L-Tyr- D-Ala- D-His- L-Lys- L-Leu- D-Ala- L-Thr- NH₂ |
| 133 | L-Tyr- L-Leu- L-Ala- L-Lys- L-Leu- D-Ala- L-Thr- OMe |
| 134 | L-Tyr- L-Leu- L-Ala- L-Lys- L-Leu- D-Ala- L-Thr- NH₂ |
| 135 | L-Tyr- L-Ala- L-Ala- L-Lys- L-Leu- D-Ala- L-Thr- OMe |
| 136 | L-Tyr- L-Ala- L-Ala- L-Lys- L-Leu- D-Ala- L-Thr- NH₂ |
| 137 | L-Tyr- D-Ala- L-Ala- L-Lys- L-Leu- D-Ala- L-Thr- OMe |
| 138 | L-Tyr- D-Ala- L-Ala- L-Lys- L-Leu- D-Ala- L-Thr- NH₂ |
| 139 | L-Tyr- L-Leu- D-Ala- L-Lys- L-Leu- D-Ala- L-Thr- OMe |
| 140 | L-Tyr- L-Leu- D-Ala- L-Lys- L-Leu- D-Ala- L-Thr- NH₂ |
| 141 | L-Tyr- L-Ala- D-Ala- L-Lys- L-Leu- D-Ala- L-Thr- OMe |
| 142 | L-Tyr- L-Ala- D-Ala- L-Lys- L-Leu- D-Ala- L-Thr- NH₂ |
| 143 | L-Tyr- D-Ala- D-Ala- L-Lys- L-Leu- D-Ala- L-Thr- OMe |
| 144 | L-Tyr- D-Ala- D-Ala- L-Lys- L-Leu- D-Ala- L-Thr- NH₂ |
| 145 | D-Thr- D-Gln- D-Leu- D-Lys- D-His- D-Leu- OMe |
| 146 | D-Thr- D-Gln- D-Leu- D-Lys- D-His- D-Leu- NH₂ |
| 147 | D-Thr- D-Gln- D-Leu- D-Lys- D-His- D-Ala- OMe |
| 148 | D-Thr- D-Gln- D-Leu- D-Lys- D-His- D-Ala- NH₂ |
| 149 | D-Thr- D-Gln- D-Leu- D-Lys- D-His- L-Ala- OMe |
| 150 | D-Thr- D-Gln- D-Leu- D-Lys- D-His- L-Ala- NH₂ |
| 151 | D-Thr- D-Gln- D-Leu- D-Lys- L-His- D-Leu- OMe |
| 152 | D-Thr- D-Gln- D-Leu- D-Lys- L-His- D-Leu- NH₂ |
| 153 | D-Thr- D-Gln- D-Leu- D-Lys- L-His- D-Ala- OMe |
| 154 | D-Thr- D-Gln- D-Leu- D-Lys- L-His- D-Ala- NH₂ |
| 155 | D-Thr- D-Gln- D-Leu- D-Lys- L-His- L-Ala- OMe |
| 156 | D-Thr- D-Gln- D-Leu- D-Lys- L-His- L-Ala- NH₂ |
| 157 | D-Thr- D-Gln- D-Leu- D-Lys- D-Ala- D-Leu- OMe |
| 158 | D-Thr- D-Gln- D-Leu- D-Lys- D-Ala- D-Leu- NH₂ |
| 159 | D-Thr- D-Gln- D-Leu- D-Lys- D-Ala- D-Ala- OMe |
| 160 | D-Thr- D-Gln- D-Leu- D-Lys- D-Ala- D-Ala- NH₂ |
| 161 | D-Thr- D-Gln- D-Leu- D-Lys- D-Ala- L-Ala- OMe |
| 162 | D-Thr- D-Gln- D-Leu- D-Lys- D-Ala- L-Ala- NH₂ |
| 163 | D-Thr- D-Gln- D-Leu- D-Lys- L-Ala- D-Leu- OMe |
| 164 | D-Thr- D-Gln- D-Leu- D-Lys- L-Ala- D-Leu- NH₂ |
| 165 | D-Thr- D-Gln- D-Leu- D-Lys- L-Ala- D-Ala- OMe |
| 166 | D-Thr- D-Gln- D-Leu- D-Lys- L-Ala- D-Ala- NH₂ |
| 167 | D-Thr- D-Gln- D-Leu- D-Lys- L-Ala- L-Ala- OMe |
| 168 | D-Thr- D-Gln- D-Leu- D-Lys- L-Ala- L-Ala- NH₂ |
| 169 | D-Thr- D-Ala- D-Leu- D-Lys- D-His- D-Leu- OMe |
| 170 | D-Thr- D-Ala- D-Leu- D-Lys- D-His- D-Leu- NH₂ |
| 171 | D-Thr- D-Ala- D-Leu- D-Lys- D-His- D-Ala- OMe |
| 172 | D-Thr- D-Ala- D-Leu- D-Lys- D-His- D-Ala- NH₂ |
| 173 | D-Thr- D-Ala- D-Leu- D-Lys- D-His- L-Ala- OMe |
| 174 | D-Thr- D-Ala- D-Leu- D-Lys- D-His- L-Ala- NH₂ |
| 175 | D-Thr- D-Ala- D-Leu- D-Lys- L-His- D-Leu- OMe |
| 176 | D-Thr- D-Ala- D-Leu- D-Lys- L-His- D-Leu- NH₂ |
| 177 | D-Thr- D-Ala- D-Leu- D-Lys- L-His- D-Ala- OMe |
| 178 | D-Thr- D-Ala- D-Leu- D-Lys- L-His- D-Ala- NH₂ |
| 179 | D-Thr- D-Ala- D-Leu- D-Lys- L-His- L-Ala- OMe |
| 180 | D-Thr- D-Ala- D-Leu- D-Lys- L-His- L-Ala- NH₂ |
| 181 | D-Thr- D-Ala- D-Leu- D-Lys- D-Ala- D-Leu- OMe |
| 182 | D-Thr- D-Ala- D-Leu- D-Lys- D-Ala- D-Leu- NH₂ |
| 183 | D-Thr- D-Ala- D-Leu- D-Lys- D-Ala- D-Ala- OMe |
| 184 | D-Thr- D-Ala- D-Leu- D-Lys- D-Ala- D-Ala- NH₂ |
| 185 | D-Thr- D-Ala- D-Leu- D-Lys- D-Ala- L-Ala- OMe |
| 186 | D-Thr- D-Ala- D-Leu- D-Lys- D-Ala- L-Ala- NH₂ |
| 187 | D-Thr- D-Ala- D-Leu- D-Lys- L-Ala- D-Leu- OMe |
| 188 | D-Thr- D-Ala- D-Leu- D-Lys- L-Ala- D-Leu- NH₂ |
| 189 | D-Thr- D-Ala- D-Leu- D-Lys- L-Ala- D-Ala- OMe |
| 190 | D-Thr- D-Ala- D-Leu- D-Lys- L-Ala- D-Ala- NH₂ |
| 191 | D-Thr- D-Ala- D-Leu- D-Lys- L-Ala- L-Ala- OMe |
| 192 | D-Thr- D-Ala- D-Leu- D-Lys- L-Ala- L-Ala- NH₂ |
| 193 | D-Thr- L-Ala- D-Leu- D-Lys- D-His- D-Leu- OMe |
| 194 | D-Thr- L-Ala- D-Leu- D-Lys- D-His- D-Leu- NH₂ |
| 195 | D-Thr- L-Ala- D-Leu- D-Lys- D-His- D-Ala- OMe |
| 196 | D-Thr- L-Ala- D-Leu- D-Lys- D-His- D-Ala- NH₂ |
| 197 | D-Thr- L-Ala- D-Leu- D-Lys- D-His- L-Ala- OMe |
| 198 | D-Thr- L-Ala- D-Leu- D-Lys- D-His- L-Ala- NH₂ |
| 199 | D-Thr- L-Ala- D-Leu- D-Lys- L-His- D-Leu- OMe |
| 200 | D-Thr- L-Ala- D-Leu- D-Lys- L-His- D-Leu- NH₂ |
| 201 | D-Thr- L-Ala- D-Leu- D-Lys- L-His- D-Ala- OMe |
| 202 | D-Thr- L-Ala- D-Leu- D-Lys- L-His- D-Ala- NH₂ |
| 203 | D-Thr- L-Ala- D-Leu- D-Lys- L-His- L-Ala- OMe |
| 204 | D-Thr- L-Ala- D-Leu- D-Lys- L-His- L-Ala- NH₂ |
| 205 | D-Thr- L-Ala- D-Leu- D-Lys- D-Ala- D-Leu- OMe |
| 206 | D-Thr- L-Ala- D-Leu- D-Lys- D-Ala- D-Leu- NH₂ |
| 207 | D-Thr- L-Ala- D-Leu- D-Lys- D-Ala- D-Ala- OMe |
| 208 | D-Thr- L-Ala- D-Leu- D-Lys- D-Ala- D-Ala- NH₂ |
| 209 | D-Thr- L-Ala- D-Leu- D-Lys- D-Ala- L-Ala- OMe |
| 210 | D-Thr- L-Ala- D-Leu- D-Lys- D-Ala- L-Ala- NH₂ |
| 211 | D-Thr- L-Ala- D-Leu- D-Lys- L-Ala- D-Leu- OMe |
| 212 | D-Thr- L-Ala- D-Leu- D-Lys- L-Ala- D-Leu- NH₂ |
| 213 | D-Thr- L-Ala- D-Leu- D-Lys- L-Ala- D-Ala- OMe |
| 214 | D-Thr- L-Ala- D-Leu- D-Lys- L-Ala- D-Ala- NH₂ |
| 215 | D-Thr- L-Ala- D-Leu- D-Lys- L-Ala- L-Ala- OMe |
| 216 | D-Thr- L-Ala- D-Leu- D-Lys- L-Ala- L-Ala- NH₂ |
| 217 | D-Thr- D-Gln- D-Leu- D-Lys- D-His- D-Leu- D-Tyr- OMe |
| 218 | D-Thr- D-Gln- D-Leu- D-Lys- D-His- D-Leu- D-Tyr- NH₂ |
| 219 | D-Thr- D-Gln- D-Leu- D-Lys- D-His- D-Ala- D-Tyr- OMe |
| 220 | D-Thr- D-Gln- D-Leu- D-Lys- D-His- D-Ala- D-Tyr- NH₂ |
| 221 | D-Thr- D-Gln- D-Leu- D-Lys- D-His- L-Ala- D-Tyr- OMe |
| 222 | D-Thr- D-Gln- D-Leu- D-Lys- D-His- L-Ala- D-Tyr- NH₂ |
| 223 | D-Thr- D-Gln- D-Leu- D-Lys- L-His- D-Leu- D-Tyr- OMe |
| 224 | D-Thr- D-Gln- D-Leu- D-Lys- L-His- D-Leu- D-Tyr- NH₂ |
| 225 | D-Thr- D-Gln- D-Leu- D-Lys- L-His- D-Ala- D-Tyr- OMe |
| 226 | D-Thr- D-Gln- D-Leu- D-Lys- L-His- D-Ala- D-Tyr- NH₂ |
| 227 | D-Thr- D-Gln- D-Leu- D-Lys- L-His- L-Ala- D-Tyr- OMe |
| 228 | D-Thr- D-Gln- D-Leu- D-Lys- L-His- L-Ala- D-Tyr- NH₂ |
| 229 | D-Thr- D-Gln- D-Leu- D-Lys- D-Ala- D-Leu- D-Tyr- OMe |
| 230 | D-Thr- D-Gln- D-Leu- D-Lys- D-Ala- D-Leu- D-Tyr- NH₂ |
| 231 | D-Thr- D-Gln- D-Leu- D-Lys- D-Ala- D-Ala- D-Tyr- OMe |
| 232 | D-Thr- D-Gln- D-Leu- D-Lys- D-Ala- D-Ala- D-Tyr- NH₂ |
| 233 | D-Thr- D-Gln- D-Leu- D-Lys- D-Ala- L-Ala- D-Tyr- OMe |
| 234 | D-Thr- D-Gln- D-Leu- D-Lys- D-Ala- L-Ala- D-Tyr- NH₂ |
| 235 | D-Thr- D-Gln- D-Leu- D-Lys- L-Ala- D-Leu- D-Tyr- OMe |
| 236 | D-Thr- D-Gln- D-Leu- D-Lys- L-Ala- D-Leu- D-Tyr- NH₂ |
| 237 | D-Thr- D-Gln- D-Leu- D-Lys- L-Ala- D-Ala- D-Tyr- OMe |
| 238 | D-Thr- D-Gln- D-Leu- D-Lys- L-Ala- D-Ala- D-Tyr- NH₂ |
| 239 | D-Thr- D-Gln- D-Leu- D-Lys- L-Ala- L-Ala- D-Tyr- OMe |
| 240 | D-Thr- D-Gln- D-Leu- D-Lys- L-Ala- L-Ala- D-Tyr- NH₂ |
| 241 | D-Thr- D-Ala- D-Leu- D-Lys- D-His- D-Leu- D-Tyr- OMe |
| 242 | D-Thr- D-Ala- D-Leu- D-Lys- D-His- D-Leu- D-Tyr- NH₂ |
| 243 | D-Thr- D-Ala- D-Leu- D-Lys- D-His- D-Ala- D-Tyr- OMe |
| 244 | D-Thr- D-Ala- D-Leu- D-Lys- D-His- D-Ala- D-Tyr- NH₂ |
| 245 | D-Thr- D-Ala- D-Leu- D-Lys- D-His- L-Ala- D-Tyr- OMe |
| 246 | D-Thr- D-Ala- D-Leu- D-Lys- D-His- L-Ala- D-Tyr- NH₂ |
| 247 | D-Thr- D-Ala- D-Leu- D-Lys- L-His- D-Leu- D-Tyr- OMe |
| 248 | D-Thr- D-Ala- D-Leu- D-Lys- L-His- D-Leu- D-Tyr- NH₂ |
| 249 | D-Thr- D-Ala- D-Leu- D-Lys- L-His- D-Ala- D-Tyr- OMe |
| 250 | D-Thr- D-Ala- D-Leu- D-Lys- L-His- D-Ala- D-Tyr- NH₂ |
| 251 | D-Thr- D-Ala- D-Leu- D-Lys- L-His- L-Ala- D-Tyr- OMe |
| 252 | D-Thr- D-Ala- D-Leu- D-Lys- L-His- L-Ala- D-Tyr- NH₂ |
| 253 | D-Thr- D-Ala- D-Leu- D-Lys- D-Ala- D-Leu- D-Tyr- OMe |
| 254 | D-Thr- D-Ala- D-Leu- D-Lys- D-Ala- D-Leu- D-Tyr- NH₂ |
| 255 | D-Thr- D-Ala- D-Leu- D-Lys- D-Ala- D-Ala- D-Tyr- OMe |
| 256 | D-Thr- D-Ala- D-Leu- D-Lys- D-Ala- D-Ala- D-Tyr- NH₂ |
| 257 | D-Thr- D-Ala- D-Leu- D-Lys- D-Ala- L-Ala- D-Tyr- OMe |
| 258 | D-Thr- D-Ala- D-Leu- D-Lys- D-Ala- L-Ala- D-Tyr- NH₂ |
| 259 | D-Thr- D-Ala- D-Leu- D-Lys- L-Ala- D-Leu- D-Tyr- OMe |
| 260 | D-Thr- D-Ala- D-Leu- D-Lys- L-Ala- D-Leu- D-Tyr- NH₂ |
| 261 | D-Thr- D-Ala- D-Leu- D-Lys- L-Ala- D-Ala- D-Tyr- OMe |
| 262 | D-Thr- D-Ala- D-Leu- D-Lys- L-Ala- D-Ala- D-Tyr- NH₂ |
| 263 | D-Thr- D-Ala- D-Leu- D-Lys- L-Ala- L-Ala- D-Tyr- OMe |
| 264 | D-Thr- D-Ala- D-Leu- D-Lys- L-Ala- L-Ala- D-Tyr- NH₂ |
| 265 | D-Thr- L-Ala- D-Leu- D-Lys- D-His- D-Leu- D-Tyr- OMe |
| 266 | D-Thr- L-Ala- D-Leu- D-Lys- D-His- D-Leu- D-Tyr- NH₂ |
| 267 | D-Thr- L-Ala- D-Leu- D-Lys- D-His- D-Ala- D-Tyr- OMe |
| 268 | D-Thr- L-Ala- D-Leu- D-Lys- D-His- D-Ala- D-Tyr- NH₂ |
| 269 | D-Thr- L-Ala- D-Leu- D-Lys- D-His- L-Ala- D-Tyr- OMe |
| 270 | D-Thr- L-Ala- D-Leu- D-Lys- D-His- L-Ala- D-Tyr- NH₂ |
| 271 | D-Thr- L-Ala- D-Leu- D-Lys- L-His- D-Leu- D-Tyr- OMe |
| 272 | D-Thr- L-Ala- D-Leu- D-Lys- L-His- D-Leu- D-Tyr- NH₂ |
| 273 | D-Thr- L-Ala- D-Leu- D-Lys- L-His- D-Ala- D-Tyr- OMe |
| 274 | D-Thr- L-Ala- D-Leu- D-Lys- L-His- D-Ala- D-Tyr- NH₂ |
| 275 | D-Thr- L-Ala- D-Leu- D-Lys- L-His- L-Ala- D-Tyr- OMe |
| 276 | D-Thr- L-Ala- D-Leu- D-Lys- L-His- L-Ala- D-Tyr- NH₂ |
| 277 | D-Thr- L-Ala- D-Leu- D-Lys- D-Ala- D-Leu- D-Tyr- OMe |
| 278 | D-Thr- L-Ala- D-Leu- D-Lys- D-Ala- D-Leu- D-Tyr- NH₂ |
| 279 | D-Thr- L-Ala- D-Leu- D-Lys- D-Ala- D-Ala- D-Tyr- OMe |
| 280 | D-Thr- L-Ala- D-Leu- D-Lys- D-Ala- D-Ala- D-Tyr- NH₂ |
| 281 | D-Thr- L-Ala- D-Leu- D-Lys- D-Ala- L-Ala- D-Tyr- OMe |
| 282 | D-Thr- L-Ala- D-Leu- D-Lys- D-Ala- L-Ala- D-Tyr- NH₂ |
| 283 | D-Thr- L-Ala- D-Leu- D-Lys- L-Ala- D-Leu- D-Tyr- OMe |
| 284 | D-Thr- L-Ala- D-Leu- D-Lys- L-Ala- D-Leu- D-Tyr- NH₂ |
| 285 | D-Thr- L-Ala- D-Leu- D-Lys- L-Ala- D-Ala- D-Tyr- OMe |
| 286 | D-Thr- L-Ala- D-Leu- D-Lys- L-Ala- D-Ala- D-Tyr- NH₂ |
| 287 | D-Thr- L-Ala- D-Leu- D-Lys- L-Ala- L-Ala- D-Tyr- OMe |
| 288 | D-Thr- L-Ala- D-Leu- D-Lys- L-Ala- L-Ala- D-Tyr- NH₂ |

What is claimed is:

1. A storage stable pharmaceutical composition comprising at least one active pharmaceutical ingredient selected from peptides of the general formula 1 [SEQ ID NO: 1-144]
H-XDL-XDL1-XDL2-L-Lys-L-Leu-XDL3-L-Thr-R2 (1),
wherein:
H—hydrogen,
XDL—the absence of an amino acid or L-Tyr,
XDL1—one of the amino acids: L-Leu, L-Ala or D-Ala,
XDL2—one of the amino acids: L-His, D-His, L-Ala or D-Ala,
XDL3—one of the amino acids: L-Gln, L-Ala or D-Ala;
R2—OMe, or NHz;
or
peptides of the general formula 2 [SEQ ID NO: 145-288]
H-D-Thr-XDL4-D-Leu-D-Lys-XDL5-XDL6-XDL7-R2 (2),
wherein:
H—hydrogen,
XDL4—one of the amino acids: D-Gln, D-Ala or L-Ala,
XDL5—one of the amino acids: D-His, L-His, D-Ala or L-Ala,
XDL6—one of the amino acids: D-Leu, D-Ala or L-Ala,
XDL7—the absence of an amino acid or D-Tyr,
R2—OMe, or NH₂;
or, their derivatives, analogs or pharmaceutically acceptable salts, or co-crystals of these peptides, their derivatives or analogues;
and a pharmaceutically acceptable carrier,
wherein the composition retains at least 80% of the potency of active pharmaceutical ingredient in the pharmaceutical composition after storage for at least four months at room temperature.

2. The storage stable pharmaceutical composition of claim 1, wherein the composition when stored at 25° C. in closed vials for four months, the total amount of related substances does not increase more than 15% by weight to the active pharmaceutical ingredient.

3. The storage stable pharmaceutical composition of claim 1, wherein the composition retains at least 80% of the potency of active pharmaceutical ingredient in the pharmaceutical composition after steam sterilization.

4. The storage stable pharmaceutical composition of claim 1, wherein the derivative is a peptide of five to fourteen amino acid residues containing an amino acid sequence that has at least 50% amino acid sequence identity in length relative to the amino acid sequence of peptides of the general formula 1 [SEQ ID NO: 1-144] or 2 [SEQ ID NO: 145-288], which retains peptide activity and/or interacting with the same specific receptor, and may differ from these sequences by truncation, deletion, substitution, addition or modification of one or more amino acids selected from a naturally occurring amino acid, a natural non-protein amino acid, a non-natural amino acid, or a modified or unusual amino acid residue.

5. The storage stable pharmaceutical composition of claim 1, wherein the pharmaceutically acceptable carrier comprises a solvent, wherein the solvent is of water, ethylene glycol, diethylene glycol, triethylene glycol, tetraethylene glycol, polyethylene glycol, propylene glycol, dipropylene glycol, tripropylene glycol, methoxypropylene glycol, polyethylene oxide, glycerin, ethanol, propyl alcohol, isopropyl alcohol, benzyl alcohol, benzyl benzoate, glycofurol, or combinations thereof.

6. The storage stable pharmaceutical composition of claim 5, wherein the pharmaceutically acceptable carrier consists entirely of a solvent that is water, ethylene glycol, diethylene glycol, triethylene glycol, tetraethylene glycol, polyethylene glycol, propylene glycol, dipropylene glycol, tripropylene glycol, methoxypropylene glycol, polyethylene oxide, glycerin, ethanol, propyl alcohol, isopropyl alcohol, benzyl alcohol, benzyl benzoate, glycofurol, or combinations thereof.

7. The storage stable pharmaceutical composition of claim 5, wherein the pharmaceutically acceptable carrier comprises at least one more ingredient that is a pH adjusting agent; isotonicity agent; preservative; chelating agent; antioxidant; surfactant; viscosifier or absorption promoter.

8. The storage stable pharmaceutical composition of claim 1, wherein the pH is from about 2.0 to about 11.0.

9. The storage stable pharmaceutical composition of claim 1, wherein the concentration of active pharmaceutical ingredient is in the range of 1 μg/ml to 500 mg/ml.

10. The storage stable pharmaceutical composition of claim 1, wherein the concentration of active pharmaceutical ingredient is in an amount sufficient to reduce the number of manifestations of pain symptoms when the pharmaceutical composition is administered to a subject in need thereof.

11. A method of treating moderate to moderately severe pain that requires analgesia at the opioid level in a subject in need thereof, comprising administering to the subject a storage stable pharmaceutical composition comprising at least one active pharmaceutical ingredient selected from peptides of the general formula 1 [SEQ ID NO: 1-144] or 2 [SEQ ID NO: 145-288], or their derivatives, or their analogues, or pharmaceutically acceptable salts and co-crystals of these peptides, their derivatives and analogues; and a pharmaceutically acceptable carrier, wherein the composition retains at least 80% of the potency of active pharmaceutical ingredient in the pharmaceutical composition after storage for at least four months at room temperature.

12. A process of preparing a storage stable pharmaceutical composition of claim 3, the process comprising, but not limited to, a steam sterilization stage that provides improved stability.
